(19) United States
(12) Patent Application Publication
(10) Pub. No.: US 2020/0308169 A1
(43) Pub. Date: Oct. 1, 2020

(54) HETEROCYCLIC COMPOUNDS AS ANTIBACTERIALS

(71) Applicant: JANSSEN SCIENCES IRELAND UNLIMITED COMPANY, Co Cork (IE)

(72) Inventors: Jérôme Émile Georges Guillemont, Andé (FR); Pierre Jean-Marie Bernard Raboisson, Wavre (BE); Abdellah Tahri, Anderlecht (BE)

(73) Assignee: JANSSEN SCIENCES IRELAND UNLIMITED COMPANY, Co Cork (IE)

(21) Appl. No.: 16/309,772

(22) PCT Filed: Jun. 15, 2017
(86) PCT No.: PCT/EP2017/064654

§ 371 Date: Dec. 13, 2018

(30) Foreign Application Priority Data
Jun. 16, 2016 (EP) 16174722.5

(51) Int. Cl. C07D 471/04; C07D 513/04; C07D 487/04; C07D 403/12; A61P 31/06

(52) U.S. Cl. CPC C07D 471/04; C07D 513/04; A61P 31/06; C07D 403/12; C07D 487/04

(57) ABSTRACT

The present invention relates to the following compounds

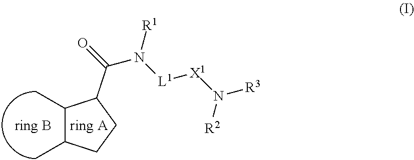

wherein the integers are as defined in the description, and where the compounds may be useful as medicaments, for instance for use in the treatment of tuberculosis.

The present invention relates to novel compounds. The invention also relates to such compounds for use as a pharmaceutical and further for the use in the treatment of bacterial diseases, including diseases caused by pathogenic mycobacteria such as Mycobacterium tuberculosis. Such compounds may work by interfering with ATP synthase in M. tuberculosis, with the inhibition of cytochrome bc₁ activity as the primary mode of action.

Hence, primarily, such compounds are antitubercular agents.

### BACKGROUND OF THE INVENTION

Mycobacterium tuberculosis is the causative agent of tuberculosis (TB), a serious and potentially fatal infection with a world-wide distribution. Estimates from the World Health Organization indicate that more than 8 million people contract TB each year, and 2 million people die from tuberculosis yearly. In the last decade, TB cases have grown 20% worldwide with the highest burden in the most impoverished communities. If these trends continue, TB incidence will increase by 41% in the next twenty years. Fifty years since the introduction of an effective chemotherapy, TB remains after AIDS, the leading infectious cause of adult mortality in the world. Complicating the TB epidemic is the rising tide of multi-drug-resistant strains, and the deadly symbiosis with HIV. People who are HIV-positive and infected with TB are 30 times more likely to develop active TB than people who are HIV-negative and TB is responsible for the death of one out of every three people with HIV/AIDS worldwide

Existing approaches to treatment of tuberculosis all involve the combination of multiple agents. For example, the regimen recommended by the U.S. Public Health Service is a combination of isoniazid, rifampicin and pyrazinamide for two months, followed by isoniazid and rifampicin alone for a further four months. These drugs are continued for a further seven months in patients infected with HIV. For patients infected with multi-drug resistant strains of M. tuberculosis, agents such as ethambutol, streptomycin, kanamycin, amikacin, capreomycin, ethionamide, cycloserine, ciprofoxacin and ofloxacin are added to the combination therapies. There exists no single agent that is effective in the clinical treatment of tuberculosis, nor any combination of agents that offers the possibility of therapy of less than six months' duration.

There is a high medical need for new drugs that improve current treatment by enabling regimens that facilitate patient and provider compliance. Shorter regimens and those that require less supervision are the best way to achieve this. Most of the benefit from treatment comes in the first 2 months, during the intensive, or bactericidal, phase when four drugs are given together; the bacterial burden is greatly reduced, and patients become noninfectious. The 4- to 6-month continuation, or sterilizing, phase is required to eliminate persisting bacilli and to minimize the risk of relapse. A potent sterilizing drug that shortens treatment to 2 months or less would be extremely beneficial. Drugs that facilitate compliance by requiring less intensive supervision also are needed. Obviously, a compound that reduces both the total length of treatment and the frequency of drug administration would provide the greatest benefit.

Complicating the TB epidemic is the increasing incidence of multi-drug-resistant strains or MDR-TB. Up to four percent of all cases worldwide are considered MDR-TB—those resistant to the most effective drugs of the four-drug standard, isoniazid and rifampin. MDR-TB is lethal when untreated and cannot be adequately treated through the standard therapy, so treatment requires up to 2 years of “second-line” drugs. These drugs are often toxic, expensive and marginally effective. In the absence of an effective therapy, infectious MDR-TB patients continue to spread the disease, producing new infections with MDR-TB strains. There is a high medical need for a new drug with a new mechanism of action, which is likely to demonstrate activity against drug resistant, in particular MDR strains.

The term “drug resistant” as used hereinbefore or hereinafter is a term well understood by the person skilled in microbiology. A drug resistant Mycobacterium is a Mycobacterium which is no longer susceptible to at least one previously effective drug; which has developed the ability to withstand antibiotic attack by at least one previously effective drug. A drug resistant strain may relay that ability to withstand to its progeny. Said resistance may be due to random genetic mutations in the bacterial cell that alters its sensitivity to a single drug or to different drugs.

MDR tuberculosis is a specific form of drug resistant tuberculosis due to a bacterium resistant to at least isoniazid and rifampicin (with or without resistance to other drugs), which are at present the two most powerful anti-TB drugs. Thus, whenever used hereinbefore or hereinafter “drug resistant” includes multi drug resistant.

Another factor in the control of the TB epidemic is the problem of latent TB. In spite of decades of tuberculosis (TB) control programs, about 2 billion people are infected by M. tuberculosis, though asymptomatically. About 10% of these individuals are at risk of developing active TB during their lifespan. The global epidemic of TB is fuelled by infection of HIV patients with TB and rise of multi-drug resistant TB strains (MDR-TB). The reactivation of latent TB is a high risk factor for disease development and accounts for 32% deaths in HIV infected individuals. To control TB epidemic, the need is to discover new drugs that can kill dormant or latent bacilli. The dormant TB can get reactivated to cause disease by several factors like suppression of host immunity by use of immunosuppressive agents like antibodies against tumor necrosis factor at or interferon-γ. In case of HIV positive patients the only prophylactic treatment available for latent TB is two-three months regimens of rifampicin, pyrazinamide. The efficacy of the treatment regime is still not clear and furthermore the length of the treatments is an important constrain in resource-limited environments. Hence there is a drastic need to identify new drugs, which can act as chemoprophylatic agents for individuals harboring latent TB bacilli.

The tubercle bacilli enter healthy individuals by inhalation; they are phagocytosed by the alveolar macrophages of the lungs. This leads to potent immune response and formation of granulomas, which consist of macrophages infected with M. tuberculosis surrounded by T cells. After a period of 6-8 weeks the host immune response cause death of infected cells by necrosis and accumulation of caseous material with certain extracellular bacilli, surrounded by macrophages, epitheloid cells and layers of lymphoid tissue at the periphery. In case of healthy individuals, most of the mycobacteria are killed in these environments but a small proportion of bacilli still survive and are thought to exist in a non-replicating, hypometabolic state and are tolerant to killing by anti-TB drugs like isoniazid. These bacilli can remain in the altered physiological environments even for individual's lifetime without showing any clinical symptoms of disease. However, in 10% of the cases these latent bacilli may reactivate to cause disease. One of the hypothesis about development of these persistent bacteria is patho-physiological environment in human lesions namely, reduced oxygen tension, nutrient limitation, and acidic pH. These factors have been postulated to render these bacteria phenotypically tolerant to major anti-mycobacterial drugs.

In addition to the management of the TB epidemic, there is the emerging problem of resistance to first-line antibiotic agents. Some important examples include penicillin-resistant Streptococcus pneumoniae, vancomycin-resistant enterococci, methicillin-resistant Staphylococcus aureus, multi-resistant salmonellae.

The consequences of resistance to antibiotic agents are severe. Infections caused by resistant microbes fail to respond to treatment, resulting in prolonged illness and greater risk of death. Treatment failures also lead to longer periods of infectivity, which increase the numbers of infected people moving in the community and thus exposing the general population to the risk of contracting a resistant strain infection.

Hospitals are a critical component of the antimicrobial resistance problem worldwide. The combination of highly susceptible patients, intensive and prolonged antimicrobial use, and cross-infection has resulted in infections with highly resistant bacterial pathogens.

Self-medication with antimicrobials is another major factor contributing to resistance. Self-medicated antimicrobials may be unnecessary, are often inadequately dosed, or may not contain adequate amounts of active drug.

Patient compliance with recommended treatment is another major problem. Patients forget to take medication, interrupt their treatment when they begin to feel better, or may be unable to afford a full course, thereby creating an ideal environment for microbes to adapt rather than be killed.

Because of the emerging resistance to multiple antibiotics, physicians are confronted with infections for which there is no effective therapy. The morbidity, mortality, and financial costs of such infections impose an increasing burden for health care systems worldwide.

Therefore, there is a high need for new compounds to treat bacterial infections, especially mycobacterial infections including drug resistant and latent mycobacterial infections, and also other bacterial infections especially those caused by resistant bacterial strains.

Anti-infective compounds for treating tuberculosis have been disclosed in e.g. international patent application WO 2011/113606. Such a document is concerned with compounds that would prevent M. tuberculosis multiplication inside the host macrophage and relates to compounds with a bicyclic core, imidazopyridines, which are linked (e.g. via an amido moiety) to e.g. an optionally substituted benzyl group.

International patent application WO 2014/015167 also discloses compounds that are disclosed as being of potential use in the treatment of tuberculosis. Such compounds disclosed herein have a bicycle (a 5,5-fused bicycle) as an essential element, which is substituted by a linker group (e.g. an amido group), which itself may be attached to another bicycle or aromatic group. Such compounds in this document do not contain a series of more than three rings.

Journal article Nature Medicine, 19, 1157-1160 (2013) by Pethe et al “Discovery of Q203, a potent clinical candidate for the treatment of tuberculosis” identifies a specific compound that was tested against M. tuberculosis. This compound Q203 is depicted below.

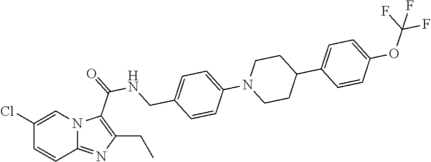 [SMILES: CCc1nc2ccc(Cl)cn2c1C(=O)NCc1ccc(N2CCC(c3ccc(OC(F)(F)F)cc3)CC2)cc1]

This clinical candidates is also discussed in journal article, J. Medicinal Chemistry, 2014, 57 (12), pp 5293-5305. It is stated to have activity against MDR tuberculosis, and have activity against the strain M. tuberculosis H37Rv at a MIC₅₀ of 0.28 nM inside macrophages. Positive control data (using known anti-TB compounds bedaquiline, isoniazid and moxifloxacin) are also reported. This document also suggests a mode of action, based on studies with mutants. It postulates that it acts by interfering with ATP synthase in M. tuberculosis, and that the inhibition of cytochrome bc₁ activity is the primary mode of action. Cytochrome bc₁ is an essential component of the electron transport chain required for ATP synthesis. It appeared that Q203 was highly active against both replicating and non-replicating bacteria International patent application WO 2015/014993 also discloses compounds as having activity against M. tuberculosis. International patent applications WO 2013/033070 and WO 2013/033167 disclose various compounds as kinase modulators. International patent applications WO 2011/057145 and WO 2016/062151 disclose various compounds stated to treat tuberculosis and to have good in vitro antituberculosis activity, respectively.

The purpose of the present invention is to provide compounds for use in the treatment of bacterial diseases, particularly those diseases caused by pathogenic bacteria such as Mycobacterium tuberculosis (including the latent disease and including drug resistant M. tuberculosis strains). Such compounds may also be novel and may act by interfering with ATP synthase in M. tuberculosis, with the inhibition of cytochrome bc₁ activity being considered the primary mode of action.

### SUMMARY OF THE INVENTION

There is now provided a compound of formula (I)

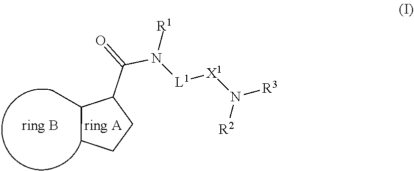

wherein
R¹ represents C₁₋₆ alkyl or hydrogen;
L¹ represents a linker group —C(R^{a})(R^{b})—;
X¹ represents an optional carbocyclic aromatic linker group (which linker group may itself be optionally substituted by one or more substituents selected from fluoro. —OH, —OC₁₋₆ alkyl and C₁₋₆ alkyl, wherein the latter two alkyl moieties are themselves optionally substituted by one or more fluoro atoms);
R^{a} and R^{b} independently represent hydrogen or C₁₋₆ alkyl (optionally substituted by one or more fluoro atoms);
R² and R³:
(i) independently represent C₁₋₆ alkyl optionally substituted by one or more substituents selected from Q and =0;
(ii) independently represent aryl or heteroaryl, each of which is optionally substituted by one or more substituents selected from Q²; or
(iii) independently represent cycloalkyl or heterocycloalkyl, each of which is optionally substituted by one or more substituents selected from Q³ and ═O;
   Q¹, Q² and Q³ each independently represent one or more substituents selected from halo, C₁₋₆ alkyl, —OC₁₋₆ alkyl (which latter two alkyl moieties may themselves be optionally substituted by one or more substituents selected from ═O and halo, e.g. fluoro, atoms), aryl and heteroaryl (which latter two aromatic groups may themselves be optionally substituted by one or more substituents selected from halo, C₁₋₆ alkyl and —OC₁₋₆ alkyl, which latter two alkyl moieties may themselves be substituted with one or more fluoro atoms);
   ring A is a 5-membered aromatic ring containing at least one heteroatom (preferably containing at least one nitrogen atom);
   ring B is a 5- or 6-membered ring, which may be aromatic or non-aromatic, optionally containing one to four heteroatoms (preferably selected from nitrogen, oxygen and sulfur);
   either ring A and/or ring B may be optionally substituted by one or more substituents selected from: halo, C₁₋₆ alkyl (optionally substituted by one or more halo, e.g. fluoro atoms) and/or —OC₁₋₆alkyl (itself optionally substituted by one or more fluoro atoms),
   or a pharmaceutically-acceptable salt thereof,
   which compounds may be referred to herein as “compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable acid addition salts as mentioned hereinabove are meant to comprise the therapeutically active non-toxic acid addition salt forms that the compounds of formula (I) are able to form. These pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

For the purposes of this invention solvates, prodrugs, N-oxides and stereoisomers of compounds of the invention are also included within the scope of the invention.

The term “prodrug” of a relevant compound of the invention includes any compound that, following oral or parenteral administration, is metabolised in vivo to form that compound in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration.

Prodrugs of compounds of the invention may be prepared by modifying functional groups present on the compound in such a way that the modifications are cleaved, in vivo when such prodrug is administered to a mammalian subject. The modifications typically are achieved by synthesising the parent compound with a prodrug substituent. Prodrugs include compounds of the invention wherein a hydroxyl, amino, sulfhydryl, carboxy or carbonyl group in a compound of the invention is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elesevier, New York-Oxford (1985).

Compounds of the invention may contain double bonds and may thus exist as E (entgegen) and Z (zusammen) geometric isomers about each individual double bond. Positional isomers may also be embraced by the compounds of the invention. All such isomers (e.g. if a compound of the invention incorporates a double bond or a fused ring, the cis- and trans-forms, are embraced) and mixtures thereof are included within the scope of the invention (e.g. single positional isomers and mixtures of positional isomers may be included within the scope of the invention).

Compounds of the invention may also exhibit tautomerism. All tautomeric forms (or tautomers) and mixtures thereof are included within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerisations. Valence tautomers include interconversions by reorganisation of some of the bonding electrons.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person.

All stereoisomers (including but not limited to diastereoisomers, enantiomers and atropisomers) and mixtures thereof (e.g. racemic mixtures) are included within the scope of the invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined. The compounds of the present invention may exist in unsolvated as well as solvated forms with pharmaceutically acceptable solvents such as water, ethanol, and the like, and it is intended that the invention embrace both solvated and unsolvated forms.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S, ¹⁸F, ³⁶Cl, ¹²³I, and ¹²⁵I. Certain isotopically-labeled compounds of the present invention (e.g., those labeled with ³H and ¹⁴C) are useful in compound and for substrate tissue distribution assays. Tritiated (³H) and carbon-14 (¹⁴C) isotopes are useful for their ease of preparation and detectability. Further, substitution with heavier isotopes such as deuterium (i.e., ²H may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the Scheme 1 and/or in the Examples herein below, by substituting an isotopically labeled reagent for a non-isotopically labeled reagent.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group). Such cycloalkyl groups may be monocyclic or bicyclic and may further be bridged. Further, when there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q} alkenyl or a C_{2-q} alkynyl group).

C_{3-q} cycloalkyl groups (where q is the upper limit of the range) that may be specifically mentioned may be monocyclic or bicyclic alkyl groups, which cycloalkyl groups may further be bridged (so forming, for example, fused ring systems such as three fused cycloalkyl groups). Such cycloalkyl groups may be saturated or unsaturated containing one or more double bonds (forming for example a cycloalkenyl group). Substituents may be attached at any point on the cycloalkyl group. Further, where there is a sufficient number (i.e. a minimum of four) such cycloalkyl groups may also be part cyclic.

The term “halo”, when used herein, preferably includes fluoro, chloro, bromo and iodo.

Heterocyclic groups when referred to herein may include aromatic or non-aromatic heterocyclic groups, and hence encompass heterocycloalkyl and hetereoaryl. Equally, “aromatic or non-aromatic 5- or 6-membered rings” may be heterocyclic groups (as well as carbocyclic groups) that have 5- or 6-members in the ring.

Heterocycloalkyl groups that may be mentioned include non-aromatic monocyclic and bicyclic heterocycloalkyl groups in which at least one (e.g. one to four) of the atoms in the ring system is other than carbon (i.e. a heteroatom), and in which the total number of atoms in the ring system is between 3 and 20 (e.g. between three and ten, e.g between 3 and 8, such as 5- to 8-). Such heterocycloalkyl groups may also be bridged. Further, such heterocycloalkyl groups may be saturated or unsaturated containing one or more double and/or triple bonds, forming for example a C_{2-q} heterocycloalkenyl (where q is the upper limit of the range) group. C_{2-q} heterocycloalkyl groups that may be mentioned include 7-azabicyclo[2.2.1]heptanyl, 6-azabicyclo[3.1.1]heptanyl, 6-azabicyclo[3.2.1]-octanyl, 8-azabicyclo-[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo-[3.2.1]octanyl, oxetanyl, oxiranyl, piperazinyl, piperidinyl, non-aromatic pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form. Heterocycloalkyl mentioned herein may be stated to be specifically monocyclic or bicyclic.

Aryl groups that may be mentioned include C₆₋₂₀, such as C₆₋₁₂ (e.g. C₆₋₁₀) aryl groups. Such groups may be monocyclic, bicyclic or tricyclic and have between 6 and 12 (e.g. 6 and 10) ring carbon atoms, in which at least one ring is aromatic. C₆₋₁₀ aryl groups include phenyl, naphthyl and the like, such as 1,2,3,4-tetrahydronaphthyl. The point of attachment of aryl groups may be via any atom of the ring system. For example, when the aryl group is polycyclic the point of attachment may be via atom including an atom of a non-aromatic ring. However, when aryl groups are polycyclic (e.g. bicyclic or tricyclic), they are preferably linked to the rest of the molecule via an aromatic ring. Most preferred aryl groups that may be mentioned herein are “phenyl”.

Unless otherwise specified, the term “heteroaryl” when used herein refers to an aromatic group containing one or more heteroatom(s) (e.g. one to four heteroatoms) preferably selected from N, O and S. Heteroaryl groups include those which have between 5 and 20 members (e.g. between 5 and 10) and may be monocyclic, bicyclic or tricyclic, provided that at least one of the rings is aromatic (so forming, for example, a mono-, bi-, or tricyclic heteroaromatic group). When the heteroaryl group is polycyclic the point of attachment may be via any atom including an atom of a non-aromatic ring. However, when heteroaryl groups are polycyclic (e.g. bicyclic or tricyclic), they are preferably linked to the rest of the molecule via an aromatic ring. Heteroaryl groups that may be mentioned include 3,4-dihydro-1H-isoquinolinyl, 1,3-dihydroisoindolyl, 1,3-dihydroisoindolyl (e.g. 3,4-dihydro-1H-isoquinolin-2-yl, 1,3-dihydroisoindol-2-yl, 1,3-dihydroisoindol-2-yl; i.e. heteroaryl groups that are linked via a non-aromatic ring), or, preferably, acridinyl, benzimidazolyl, benzodioxanyl, benzodioxepinyl, benzodioxolyl (including 1,3-benzodioxolyl), benzofuranyl, benzofurazanyl, benzothiadiazolyl (including 2,1,3-benzothiadiazolyl), benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. Heteroaryl groups mentioned herein may be stated to be specifically monocyclic or bicyclic. When heteroaryl groups are polycyclic in which there is a non-aromatic ring present, then that non-aromatic ring may be substituted by one or more ═O group. Most preferred heteroaryl groups that may be mentioned herein are 5- or 6-membered aromatic groups containing 1, 2 or 3 heteroatoms (e.g. preferably selected from nitrogen, oxygen and sulfur).

It may be specifically stated that the heteroaryl group is monocyclic or bicyclic. In the case where it is specified that the heteroaryl is bicyclic, then it may consist of a five-, six- or seven-membered monocyclic ring (e.g. a monocyclic heteroaryl ring) fused with another five-, six- or seven-membered ring (e.g. a monocyclic aryl or heteroaryl ring).

Heteroatoms that may be mentioned include phosphorus, silicon, boron and, preferably, oxygen, nitrogen and sulfur.

When “aromatic” groups are referred to herein, they may be aryl or heteroaryl. When “aromatic linker groups” are referred to herein, they may be aryl or heteroaryl, as defined herein, are preferably monocyclic (but may be polycyclic) and attached to the remainder of the molecule via any possible atoms of that linker group. However, when, specifically carbocylic aromatic linker groups are referred to, then such aromatic groups may not contain a heteroatom, i.e. they may be aryl (but not heteroaryl).

For the avoidance of doubt, where it is stated herein that a group may be substituted by one or more substituents (e.g. selected from C₁₋₆ alkyl), then those substituents (e.g. alkyl groups) are independent of one another. That is, such groups may be substituted with the same substituent (e.g. same alkyl substituent) or different (e.g. alkyl) substituents.

For the avoidance of doubt, where it is indicated that R² and R³ may independently represent substituents defined by (i), (ii) or (iii), this means that R² may represent any of the substituents defined by (i), (ii) or (iii) and that R³ is independent of R² and may at the same time represent any one of the substituents defined by (i), (ii) or (iii). Hence, for example, R² may represent a substituent defined by (i) and R³ may represent a substituent defined by (iii).

All individual features (e.g. preferred features) mentioned herein may be taken in isolation or in combination with any other feature (including preferred feature) mentioned herein (hence, preferred features may be taken in conjunction with other preferred features, or independently of them).

The skilled person will appreciate that compounds of the invention that are the subject of this invention include those that are stable. That is, compounds of the invention include those that are sufficiently robust to survive isolation from e.g. a reaction mixture to a useful degree of purity.

Certain (e.g. preferred) aspects of compounds of the invention include those in which:

R¹ represents hydrogen;
R^{a} and R^{b} independently represent hydrogen;
L¹ represents —CH₂—;
when X¹ is present, then it represents a carbocyclic aromatic linker group, for example a phenyl group or a bicyclic (carbocyclic) aromatic linker group (in which at least one of the rings of the bicycle is aromatic), for instance such that the bicycle consists of two separate rings fused with each other, in which each ring is 5- or 6-membered so forming a 6,6-, 5,6- or 5,5-fused bicyclic ring), hence including groups such as phenyl, naphthyl (including fully aromatic naphthyl and 1,2,3,4-tetrahydronaphthyl) and the like, so forming e.g. in particular:
-phenylene- (especially a 1,4-phenylene), e.g.:

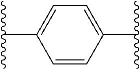 [SMILES: *c1ccc(*)cc1]

-naphthylene, e.g.:

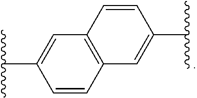 [SMILES: *c1ccc2cc(*)ccc2c1]

Such linker groups that X¹ may represent (e.g. phenylene) may be optionally substituted (e.g. by one or more substituents selected from fluoro, CH₃, CF₃, —OCH₃ and —OCF₃). In an embodiment such linker groups that X¹ may represent are unsubstituted.

Further aspects of the invention that may be mentioned include those in which:

R² and R³:
(i) independently represent C₁₋₃ alkyl optionally substituted by one or more substituents selected from Q¹ and ═O;
(ii) independently represent cycloalkyl or heterocycloalkyl (e.g. a 4-6-membered ring containing a nitrogen atom, so forming e.g. an azetidinyl group), each of which is optionally substituted by one or more substituents selected from Q³ and ═O; and/or
   Q¹, Q² and Q³ each independently represent one or more substituents selected from:
aryl (e.g. phenyl) optionally substituted by one or more substituents selected from halo, C₁₋₆ alkyl and —OC₁₋₆ alkyl (which latter two alkyl moieties may themselves be substituted with one or more fluoro atoms)
heteroaryl (e.g. a 5- or 6-membered heteroaryl group containing one or two heteroatoms, so forming e.g. a pyridinyl or thiazolyl group) optionally substituted as defined herein (but in an aspect, such heteroaryl groups are unsubstituted)
C₁₋₆ alkyl (e.g. C₁₋₃ alkyl) optionally substituted by one or more substituents selected from ═O and fluoro (e.g. so forming a —C(O)—CF₃ group)

In a major aspect of the invention, there is provided compounds of the invention in which:

one of R² and R³ represents:
cycloalkyl or heterocycloalkyl (e.g. a 4-6-membered ring containing a nitrogen atom, so forming e.g. an azetidinyl group), each of which is optionally substituted by one or more substituents selected from Q³ and ═O; and
the other (one of R² or R³) represents C₁₋₆ (e.g. C₁₋₃ alkyl) optionally substituted by one or more substituents selected from Q¹ and ═O.

In a further aspect of the invention, there is provided compounds of the invention in which:

when R² or R³ represents cycloalkyl or heterocycloalkyl, then such cyclic groups are substituted by at least one substituent selected from Q³;
Q³ represents aryl or heteroaryl, both of which are optionally substituted as defined herein.

It is preferred that compounds of the invention comprise:

ring A, which is an aromatic ring containing at least one to three (e.g. one or two) heteroatoms, preferably contains at least one nitrogen atom;
ring B is more preferably also an aromatic ring (e.g. a 5- or especially a 6-membered aromatic ring), preferably containing at least one nitrogen atom.

It is preferred that Ring A of the compounds of the invention are represented as follows:

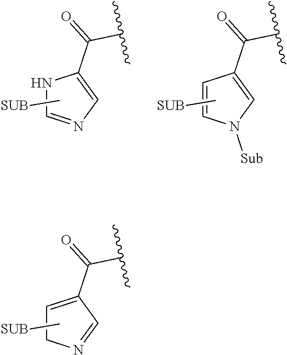

Other preferred ring A moieties include:

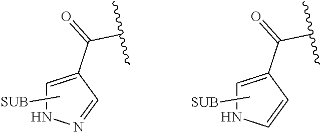

Monocyclic heteroaryl groups that may be mentioned include 5- or 6-membered rings containing one to four heteroatoms (preferably selected from nitrogen, oxygen and sulfur). It is preferred that Ring B of the compounds of the invention are represented as follows:

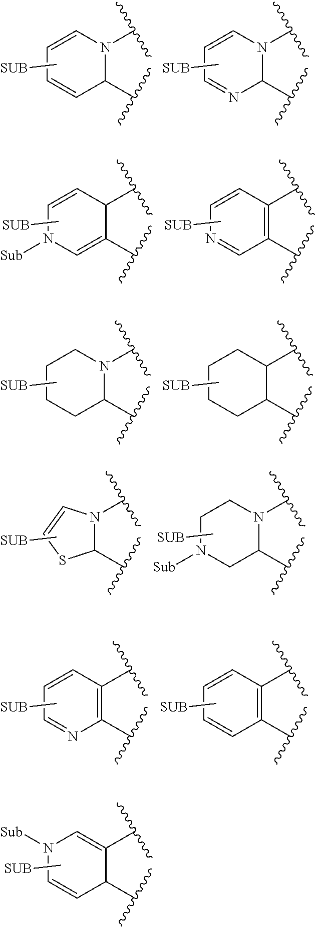

where “SUB” may be a relevant optional substituent (or more than when relevant substituent, where possible) on a carbon atom or, where possible, on a heteroatom e.g. on a NH, thus replacing the H.

Other preferred “Ring B” moieties include:

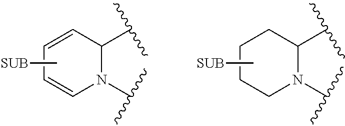

Preferred substituents (when present; e.g such optional substituents may be absent or there may be one) on ring B include C₁₋₃ alkyl (e.g. methyl) or halo (e.g. bromo or, more preferably, chloro). Other preferred substituents on ring B include —OC₁₋₆alkyl (e.g. —OC₁₋₃alkyl, such as —OCH₃).

Preferred substituents (when present; e.g such optional substituents may be absent or there may be one) on ring B include C₁₋₃ alkyl (e.g. methyl) or halo (e.g. bromo or, more preferably, chloro). Preferred substituents (when present; preferably, there may be one or two substituents) on ring A include C₁₋₃ alkyl (e.g. methyl or ethyl). When L² represents an aromatic group (e.g. phenyl or pyridyl) and such groups are substituted, preferred substituents include halo and especially —OC₁₋₃ alkyl (e.g. —O-methyl), where the latter is substituted by fluoro, so forming for example a —OCF₃ group.

The combined ring systems, i.e. Ring A and Ring B may be represented as follows:

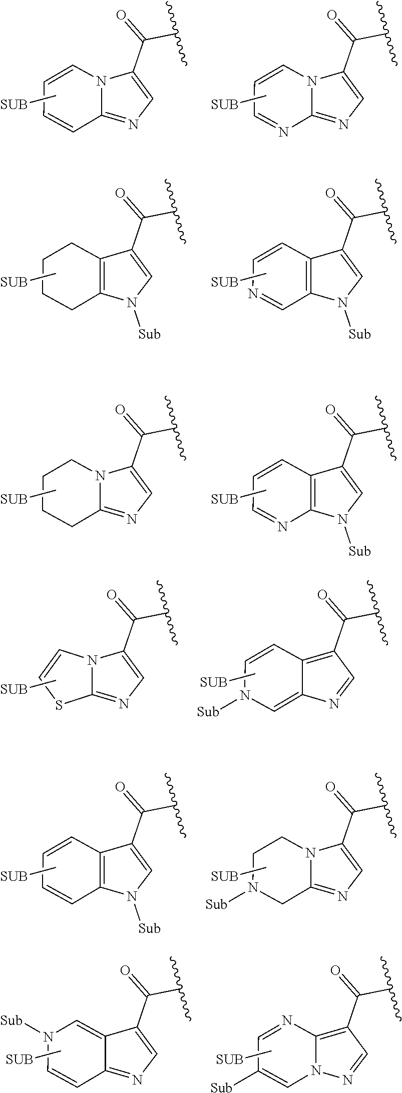

where “SUB” represents one or more possible substituents on the bicycle (i.e. on ring A and/or on ring B) and “Sub” represents a possible optional substituent on the N atom of the bicycle (unsubstituted in this context would mean “NH”).

Other combined ring A and ring B systems that may be mentioned include the following:

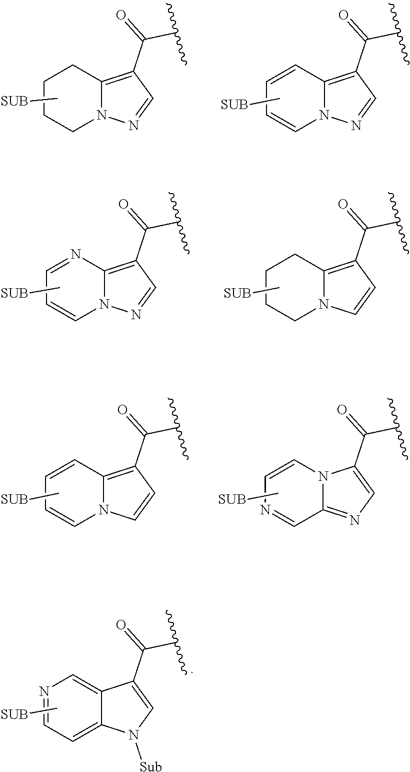

Certain compounds of the invention are mentioned (e.g. hereinbefore) for use in the treatment of tuberculosis. Certain of such compounds mentioned herein may also be novel per se. And certain of such compounds mentioned herein may be novel as medicaments/pharmaceuticals (or novel as a component of a pharmaceutical composition/formulation). Hence, in further aspects of the invention, there is provided the following compounds per se or following compounds for use as pharmaceuticals/medicaments (in the latter case such compounds may be components of a pharmaceutical composition/formulation):
(I) Compounds of formula (I) as hereinbefore defined and in which:
   L¹ represents —CH₂—;
one of R² and R³ represents:
   cycloalkyl or heterocycloalkyl (e.g. a 4-6-membered ring containing a nitrogen atom, so forming e.g. an azetidinyl group), each of which is optionally substituted by one or more substituents selected from Q³ and ═O; and
   the other (one of R² or R³) represents C₁₋₆ (e.g. C₁₋₃ alkyl) optionally substituted by one or more substituents selected from Q¹ and ═O;
(II) Compounds of formula (I) as hereinbefore defined (e.g. at (I) above) and in which:
   when R² or R³ represents cycloalkyl or heterocycloalkyl, then such cyclic groups are substituted by at least one substituent selected from Q³;
   Q³ represents aryl or heteroaryl, both of which are optionally substituted as defined herein;
   ring A and ring B together represent a 8 or 9-membered bicyclic ring (ring A is a 5-membered ring and ring B may be a 5 or 6-membered ring, in which both rings are preferably aromatic) containing at least one nitrogen atom (and in a major embodiment, at least one nitrogen atom that is common to both rings); optional substituents on ring A and ring B are halo, C₁₋₃ alkyl and —OC₁₋₃ alkyl; and
   other integers are as defined herein; and/or
(III) Compounds as hereinbefore defined (e.g. at (I) and/or (II) above) and further in which the ring A and ring B bicycles are represented as defined herein or more particularly as follows:

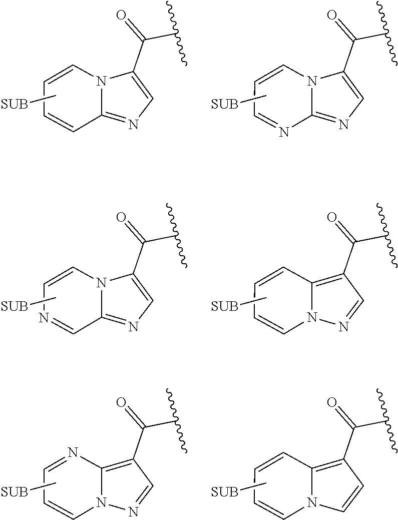

(or any one of the above-mentioned representations).

### Pharmacology

The compounds according to the invention have surprisingly been shown to be suitable for the treatment of a bacterial infection including a mycobacterial infection, particularly those diseases caused by pathogenic mycobacteria such as Mycobacterium tuberculosis (including the latent and drug resistant form thereof). The present invention thus also relates to compounds of the invention as defined hereinabove, for use as a medicine, in particular for use as a medicine for the treatment of a bacterial infection including a mycobacterial infection.

Such compounds of the invention may act by interfering with ATP synthase in M. tuberculosis, with the inhibition of cytochrome bc₁ activity being the primary mode of action. Cytochrome bc₁ is an essential component of the electron transport chain required for ATP synthesis.

Further, the present invention also relates to the use of a compound of the invention, as well as any of the pharmaceutical compositions thereof as described hereinafter for the manufacture of a medicament for the treatment of a bacterial infection including a mycobacterial infection.

Accordingly, in another aspect, the invention provides a method of treating a patient suffering from, or at risk of, a bacterial infection, including a mycobacterial infection, which comprises administering to the patient a therapeutically effective amount of a compound or pharmaceutical composition according to the invention.

The compounds of the present invention also show activity against resistant bacterial strains.

Whenever used hereinbefore or hereinafter, that the compounds can treat a bacterial infection it is meant that the compounds can treat an infection with one or more bacterial strains.

The invention also relates to a composition comprising a pharmaceutically acceptable carrier and, as active ingredient, a therapeutically effective amount of a compound according to the invention. The compounds according to the invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, in particular, for administration orally or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations.

Depending on the mode of administration, the pharmaceutical composition will preferably comprise from 0.05 to 99% by weight, more preferably from 0.1 to 70% by weight, even more preferably from 0.1 to 50% by weight of the active ingredient(s), and, from 1 to 99.95% by weight, more preferably from 30 to 99.9% by weight, even more preferably from 50 to 99.9% by weight of a pharmaceutically acceptable carrier, all percentages being based on the total weight of the composition.

The pharmaceutical composition may additionally contain various other ingredients known in the art, for example, a lubricant, stabilising agent, buffering agent, emulsifying agent, viscosity-regulating agent, surfactant, preservative, flavouring or colorant.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

The daily dosage of the compound according to the invention will, of course, vary with the compound employed, the mode of administration, the treatment desired and the mycobacterial disease indicated. However, in general, satisfactory results will be obtained when the compound according to the invention is administered at a daily dosage not exceeding 1 gram, e.g. in the range from 10 to 50 mg/kg body weight.

Given the fact that the compounds of formula (Ia) or Formula (Ib) are active against bacterial infections, the present compounds may be combined with other antibacterial agents in order to effectively combat bacterial infections.

Therefore, the present invention also relates to a combination of (a) a compound according to the invention, and (b) one or more other antibacterial agents.

The present invention also relates to a combination of (a) a compound according to the invention, and (b) one or more other antibacterial agents, for use as a medicine.

The present invention also relates to the use of a combination or pharmaceutical composition as defined directly above for the treatment of a bacterial infection.

A pharmaceutical composition comprising a pharmaceutically acceptable carrier and, as active ingredient, a therapeutically effective amount of (a) a compound according to the invention, and (b) one or more other antibacterial agents, is also comprised by the present invention.

The weight ratio of (a) the compound according to the invention and (b) the other antibacterial agent(s) when given as a combination may be determined by the person skilled in the art. Said ratio and the exact dosage and frequency of administration depends on the particular compound according to the invention and the other antibacterial agent(s) used, the particular condition being treated, the severity of the condition being treated, the age, weight, gender, diet, time of administration and general physical condition of the particular patient, the mode of administration as well as other medication the individual may be taking, as is well known to those skilled in the art. Furthermore, it is evident that the effective daily amount may be lowered or increased depending on the response of the treated subject and/or depending on the evaluation of the physician prescribing the compounds of the instant invention. A particular weight ratio for the present compound of the invention and another antibacterial agent may range from 1/10 to 10/1, more in particular from 1/5 to 5/1, even more in particular from 1/3 to 3/1.

The compounds according to the invention and the one or more other antibacterial agents may be combined in a single preparation or they may be formulated in separate preparations so that they can be administered simultaneously, separately or sequentially. Thus, the present invention also relates to a product containing (a) a compound according to the invention, and (b) one or more other antibacterial agents, as a combined preparation for simultaneous, separate or sequential use in the treatment of a bacterial infection.

The other antibacterial agents which may be combined with the compounds of the invention are for example antibacterial agents known in the art. For example, the compounds of the invention may be combined with antibacterial agents known to interfere with the respiratory chain of Mycobacterium tuberculosis, including for example direct inhibitors of the ATP synthase (e.g. bedaquiline, bedaquiline fumarate or any other compounds that may have be disclosed in the prior art, e.g. compounds disclosed in WO2004/011436), inhibitors of ndh2 (e.g. clofazimine) and inhibitors of cytochrome bd. Additional mycobacterial agents which may be combined with the compounds of the invention are for example rifampicin (=rifampin); isoniazid; pyrazinamide; amikacin; ethionamide; ethambutol; streptomycin; para-aminosalicylic acid; cycloserine; capreomycin; kanamycin; thioacetazone; PA-824; delamanid; quinolones/fluoroquinolones such as for example moxifloxacin, gatifloxacin, ofloxacin, ciprofloxacin, sparfloxacin; macrolides such as for example clarithromycin, amoxycillin with clavulanic acid; rifamycins; rifabutin; rifapentin; as well as others, which are currently being developed (but may not yet be on the market; see e.g. http://www.newtbdrugs.org/pieline.php).

### General Preparation

The compounds according to the invention can generally be prepared by a succession of steps, each of which may be known to the skilled person or described herein.

### Experimental Part

Compounds of formula I may be prepared in accordance with the techniques employed in the examples hereinafter (and those methods know by those skilled in the art), for example by using the following techniques.

Compounds of formula (I) may be prepared by:

(i) reaction of a compound of formula (II),

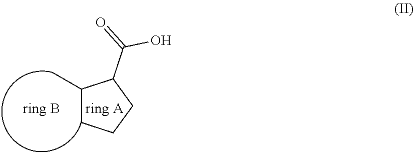

wherein the integers are as hereinbefore defined, or a suitable derivative thereof, such as a carboxylic acid ester derivative, with a compound of formula (III)

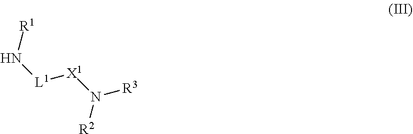

wherein the integers are as hereinbefore defined, under amide coupling reaction conditions, for example in the presence of a suitable coupling reagent (e.g. 1,1′-carbonyldiimidazole, N,N′-dicyclohexylcarbodiimide, 1-(3-dimethylaminopropyl)-3-ethylcarbodiimide (or hydrochloride thereof) or N,N′-disuccinimidyl carbonate), optionally in the presence of a suitable base (e.g. sodium hydride, sodium bicarbonate, potassium carbonate, pyridine, triethylamine, dimethylaminopyridine, diisopropylamine, sodium hydroxide, potassium tert-butoxide and/or lithium diisopropylamide (or variants thereof) and an appropriate solvent (e.g. tetrahydrofuran, pyridine, toluene, dichloromethane, chloroform, acetonitrile, dimethylformamide, trifluoromethylbenzene, dioxane or triethylamine). Alternatively, the carboxylic acid group of the compound of formula (IV) may first be converted under standard conditions to the corresponding acyl chloride (e.g. in the presence of POCl₃, PCl₅, SOCl₂ or oxalyl chloride), which acyl chloride is then reacted with a compound of formula (V), for example under similar conditions to those mentioned above;
(ii) coupling of a compound of formula (IV),

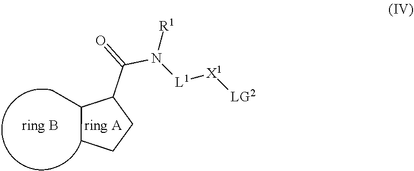

wherein the integers are as hereinbefore defined, and LG² represents a suitable leaving group, such as iodo, bromo, chloro or a sulfonate group (for example a type of group that may be deployed for a coupling), with a compound of formula (V),

HN(R²)(R³) (V)

wherein the integers are as hereinbefore defined, under standard conditions, for example optionally in the presence of an appropriate metal catalyst (or a salt or complex thereof) such as Pd(dba)₂, Pd(OAc)₂, Cu, Cu(OAc)₂, CuI, NiCl₂ or the like, with an optional additive such as Ph₃P, X-phos or the like, in the presence of an appropriate base (e.g. t-BuONa, or the like) in a suitable solvent (e.g. dioxane or the like) under reaction conditions known to those skilled in the art.

Other steps that may be mentioned include:
nucleophilic aromatic substitution reactions
other coupling reactions e.g. in which one compound contains a suitable leaving group such as one described hereinbefore with respect to LG² (and may particularly represent chloro, bromo or iodo), with another compound comprising a mutually compatible “leaving group” or another suitable group such as —B(OH)₂, —B(OR^{wx})₂ or —SN(R^{wx})₃, in which each R^{wx} independently represents a C₁₋₆ alkyl group, or, in the case of —B(OR^{wx})₂, the respective R^{wx} groups may be linked together to form a 4- to 6-membered cyclic group, thereby forming e.g. a pinacolato boronate ester group (or may represent iodo, bromo or chloro, provided that the “leaving groups” are mutually compatible), and wherein the reaction may be performed in the presence of a suitable catalyst system, e.g. a metal (or a salt or complex thereof) such as Pd, CuI, Pd/C, PdCl₂, Pd(OAc)₂, Pd(Ph₃P)₂Cl₂, Pd(Ph₃P)₄, Pd₂(dba)₃ and/or NiCl₂ (or the like) and a ligand such as PdCl₂(dppf).DCM, t-Bu₃P, (C₆H₁₁)₃P, Ph₃P or the like, in a suitable solvent and under reaction conditions known to those skilled in the art.

It is evident that in the foregoing and in the following reactions, the reaction products may be isolated from the reaction medium and, if necessary, further purified according to methodologies generally known in the art, such as extraction, crystallization and chromatography. It is further evident that reaction products that exist in more than one enantiomeric form, may be isolated from their mixture by known techniques, in particular preparative chromatography, such as preparative HPLC, chiral chromatography. Individual diastereoisomers or individual enantiomers can also be obtained by Supercritical Fluid Chromatography (SCF).

The starting materials and the intermediates are compounds that are either commercially available or may be prepared according to conventional reaction procedures generally known in the art.

### EXAMPLES

### Synthesis of Compound 1

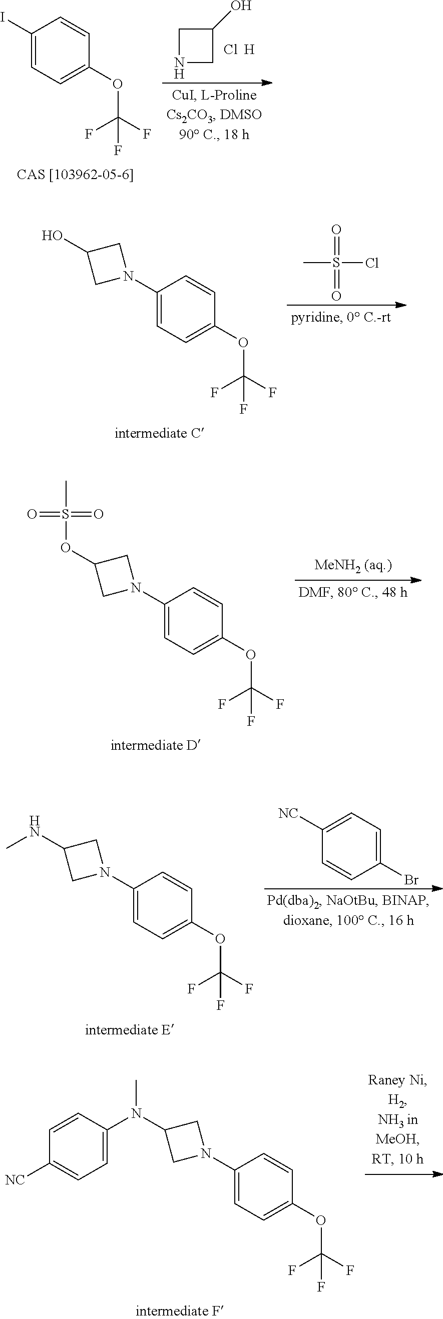

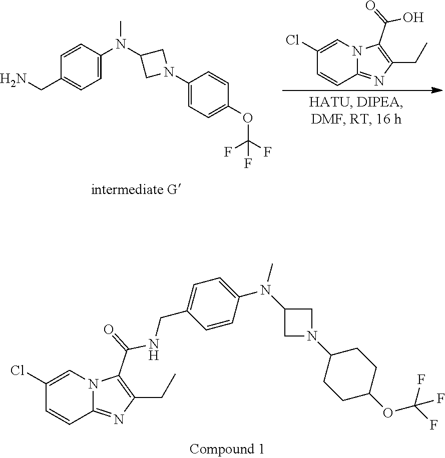 [SMILES: CN(c1ccc(CN)cc1)C1CN(c2ccc(OC(F)(F)F)cc2)C1 ; CCc1nc2ccc(Cl)cn2c1C(=O)O ; CCc1nc2ccc(Cl)cn2c1C(=O)NCc1ccc(N(C)C2CN(C3CCC(OC(F)(F)F)CC3)C2)cc1]

### Preparation of Intermediate C′

To a solution of 1-iodo-4-(trifluoromethoxy)benzene (CAS [103962-05-6], 4.9 g, 17.01 mmol) in DMSO (30 mL) was added 3-azetidin-3-ol hydrogen chloride salt (1.24 g, 11.34 mmol), cesium carbonate (9.24 g, 28.36 mmol), Copper Iodide (434 mg, 2.27 mmol) and L-proline (522 mg, 4.54 mmol) and then the mixture was heated at 90° C. for 18 h under argon atmosphere. The solution was diluted with ethyl acetate and water and the organic layer was washed with brine three times, concentrated under reduced pressure and purified by column chromatography over silica gel (petroleum ether/ethyl acetate=8:1) to give intermediate C′ as a yellow solid, 2 g, 77%.

### Preparation of intermediate D′

A solution of intermediate C′ (1.8 g, 7.72 mmol) in pyridine (20 mL) was cooled to 0° C., treated with methanesulfonyl chloride (1.76 g, 15.36 mmol). The reaction was warmed to room temperature and stirred for 3 hours. The mixture was partitioned between ethyl acetate (50 mL) and H₂O (30 mL), and the organic layer was washed with H₂O and brine, dried (Na₂SO₄), and concentrated to provide the crude product intermediate D′, 2.1 g, 87%.

### Preparation of intermediate E′

Intermediate D′ (2.1 g, 6.75 mmol) was taken up in DMF (50 mL) and treated with methyl amine (40% in H₂O, 90 mL), and the reaction was stirred at 80° C. for 48 hours. After cooling to room temperature, the mixture was partitioned between H₂O (50 mL) and ethyl acetate (100 ml). The organic layer washed with brine, dried (Na₂SO₄), concentrated under reduced pressure. The residue was purified by column chromatography over silica gel dichloromethane/methanol (15:1) to give intermediate E′, 0.5 g, 30%.

### Preparation of Intermediate F′

A mixture of intermediate E′ (0.75 g, 3.04 mmol), 4-bromobenzonitrile (CAS [623-00-7], 0.554 g, 3.04 mmol), NaOtBu (1.46 g, 15.2 mmol) and Xphos (0.29 g, 0.609 mmol) in dioxane (10 mL) was stirred at room temperature for 20 min under nitrogen flow. Then to the stirring solution was added Pd(dba)₂ (0.175 g, 0.305 mmol) and stirred for 10 min under nitrogen flow. The mixture was irradiated in microwave at 110° C. for 1 h. The crude mixture was filtered over Celite® and the solvent was evaporated. The residue was purified by high performance liquid chromatography (Phenomenex Gemini C18 250×50 mm×10 μm, 90 ml/min, mobile phase: water (containing 0.05% NH₃H₂O)/acetonitrile, gradient from 70/30 to 30/70). The desired fraction was collected and evaporated to remove off acetonitrile in vacuum. The residue was lyophilized to afford intermediate F′, 0.3 g, 21%

### Preparation of Intermediate G′

To a solution of intermediate F′ (0.2 g, 0.645 mmol) in ammonia 7M in MeOH (10 mL) was added Raney Ni (0.1 g) under N₂. The suspension was degassed under vacuum and purged with H₂ several times. The mixture was stirred under H₂ (15 psi) at 25° C. for 10 hours. The suspension was filtered through a pad of Celite® was washed with methanol (40 mL). The combined filtrates were concentrated to dryness to give intermediate G′, 0.21 g, 99%.

### Preparation of Compound 1

To a solution of 6-chloro-2-ethylimidazo[3,2-a]pyridine-3-carboxylic acid CAS [1216142-18-5], 0.19 g, 0.85 mmol) in DMF (30 mL) was added intermediate G′ (0.27 g, 0.768 mmol), HATU (0.35 g, 0.92 mmol) and diisopropylethylamine (0.28 g, 2.31 mmol). The mixture was stirred at room temperature overnight. The mixture was diluted with water (30 mL) and extracted with ethyl acetate (20 mL×3). The organic layers were dried over Na₂SO₄, filtered and concentrated in vacuum. The residue was purified by high performance liquid chromatography (Waters Xbridge Prep OBD C18 150×30×5μ, 25 ml/min, mobile phase: water (containing 0.05% NH₃.H₂O)/acetonitrile, Gradient: from 40/60 to 10/90). The desired fraction was collected and evaporated to remove off acetonitrile in vacuum. The residue was lyophilized to give Compound 1, 0.297 g, 66%. ¹H NMR (400 MHz, CHLOROFORM-d) δ=9.53 (d, J=1.3 Hz, 1H), 7.54 (d, J=9.7 Hz, 1H), 7.32-7.26 (m, 3H), 7.07 (d, J=8.4 Hz, 2H), 6.77 (d, J=8.8 Hz, 2H), 6.43 (d, J=8.8 Hz, 2H), 6.04 (br. s., 1H), 4.61 (d, J=5.7 Hz, 2H), 4.50 (quin, J=6.4 Hz, 1H), 4.20 (t, J=7.3 Hz, 2H), 3.86-3.79 (m, 2H), 3.01-2.91 (m, 5H), 1.40 (t, J=7.5 Hz, 3H).

### Synthesis of Compound 2

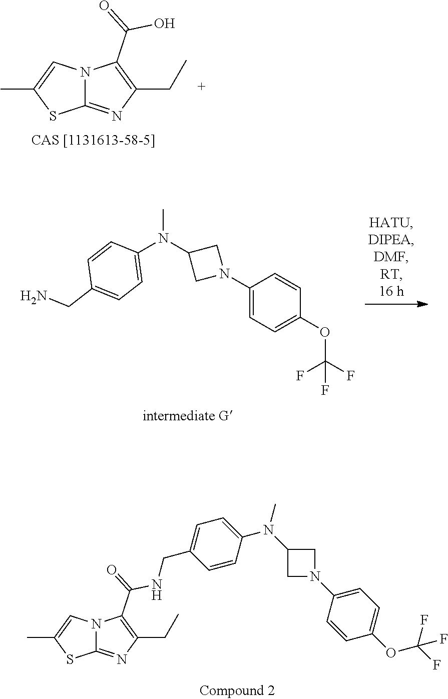 [SMILES: CCc1nc2sc(C)cn2c1C(=O)O ; CN(c1ccc(CN)cc1)C1CN(c2ccc(OC(F)(F)F)cc2)C1 ; CCc1nc2sc(C)cn2c1C(=O)NCc1ccc(N(C)C2CN(c3ccc(OC(F)(F)F)cc3)C2)cc1]

To a solution of intermediate G′ (0.09 g, 0.256 mmol) in CH₂Cl₂ (20 mL) was added 6-Ethyl-2-methylimidazo[2,1-b]thiazole-5-carboxylic acid (CAS [1131613-58-5, 0.054 g, 0.256 mmol), HATU (0.127 g, 0.333 mmol) and diisopropylethylamine (0.099 g, 0.768 mmol). The mixture was stirred at room temperature overnight. The mixture was diluted with water (20 mL) and extracted with dichloromethane (10 mL×3). The organic layers were dried over Na₂SO₄, filtered and concentrated in vacuum. The residue was purified by high performance liquid chromatography (YMC-Actus Triart C18 150×30×5μ, 25 ml/min, mobile phase: water (containing 0.05% NH₃.H₂O)/Acetonitrile, gradient from 29/71 to 0/100). The desired fraction was collected and evaporated to remove off acetonitrile in vacuum. The residue was lyophilized to give Compound 2, 0.101 g, 73%.

### Synthesis of Compound 3

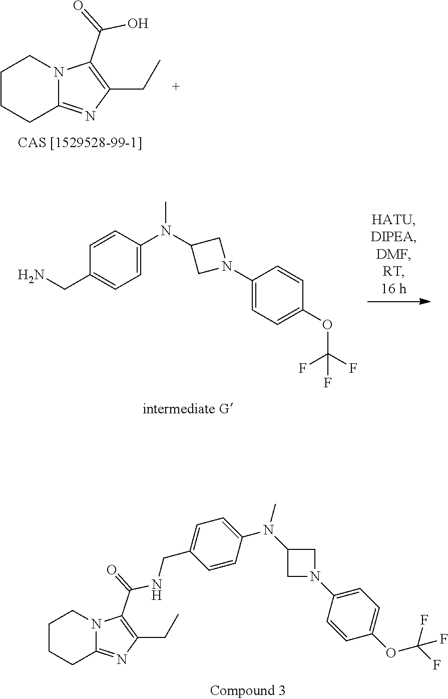 [SMILES: CCc1nc2n(c1C(=O)O)CCCC2 ; CN(c1ccc(CN)cc1)C1CN(c2ccc(OC(F)(F)F)cc2)C1 ; CCc1nc2n(c1C(=O)NCc1ccc(N(C)C3CN(c4ccc(OC(F)(F)F)cc4)C3)cc1)CCCC2]

To a solution of intermediate G′ (0.13 g, 0.370 mmol) in DMF (20 mL) was added 2-ethyl-5H,6H,7H,8H-imidazo[1.2-a]pyridine-3-carboxylic acid (CAS [1529528-99-1], 0.072 g, 0.370 mmol), HATU (0.183 g, 0.481 mmol) and diisopropylethylamine (0.144 g, 1.11 mmol). The mixture was stirred at room temperature overnight. The mixture was diluted with water (20 mL) and extracted with ethyl acetate (10 mL×3). The organic layers were dried over Na₂SO₄, filtered and concentrated in vacuum. The residue was purified by high performance liquid chromatography (YMC-Actus Triart C18 150×30×5μ, 25 ml/min, mobile phase: water (containing 0.05% NH₃.H₂O)/Acetonitrile, gradient from 44/56 to 14/86). The desired fraction was collected and evaporated to remove off acetonitrile in vacuum. The residue was lyophilized to give Compound 3, 0.066 g, 34%.

### Synthesis of Compound 4

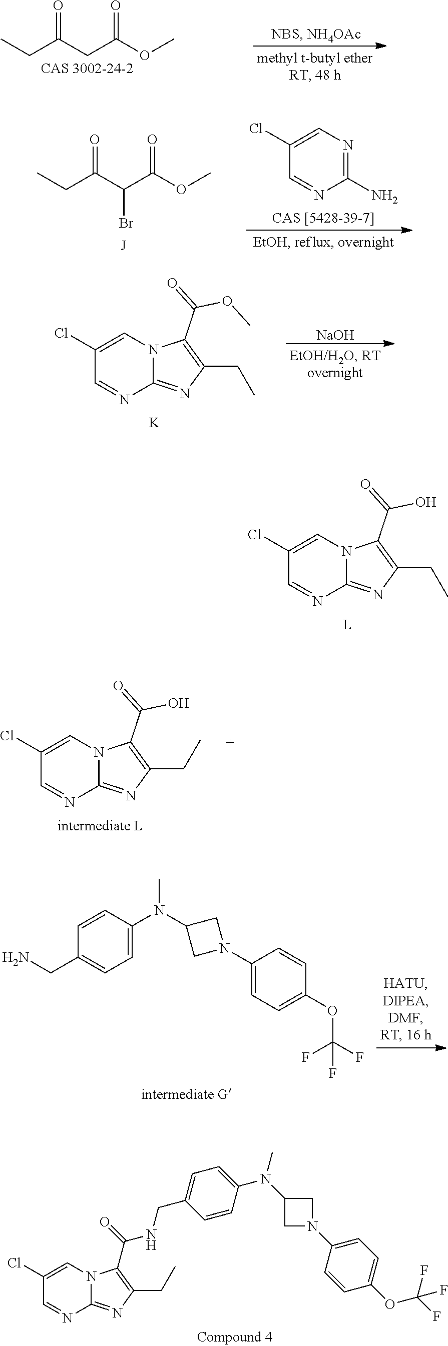

### Preparation of Intermediate J

NBS (45.1 g, 254 mmol) and NH₄OAc (5.33 g, 69.2 mmol) were added to a solution of methyl-3-oxovalerate (CAS[30414-53-0], 30 g, 231 mmol) in methyl t-butylether (600 mL). The mixture was stirred at room temperature for 48 h. The mixture was filtered and washed with H₂O, dried over Na₂SO₄ and filtered. The filtrate was concentrated under vacuum. The residue was purified by column chromatography over silica gel (eluent: petroleum ether/ethyl acetate 20/1) to give intermediate J (20.0 g, yield: 35%).

### Preparation of intermediate K

A solution of 5-Chloro-2-pyridinamine (CAS [5428-89-7], 12.0 g, 93.0 mmol) and intermediate J (25.0 g, 112 mmol) in ethanol (60 mL) was refluxed overnight. The mixture was concentrated under vacuum. The residue was dissolved into ethyl acetate (100 mL). The solution was washed with water (2×100 mL), brine (100 mL), dried over sodium sulfate, filtered and concentrated under vacuum. The residue was purified by column chromatography over silica gel (eluent: petroleum ether/ethyl acetate 3/1) to give intermediate K (700 mg, yield: 3%).

### Preparation of Intermediate L

A mixture of intermediate K (700 mg, 2.10 mmol) and sodium hydroxide (252 mg, 6.30 mmol) in ethanol (2 ml) and H₂O (2 mL) was stirred overnight at room temperature. Water (20 mL) was added and the solution was acidified with 2 M aqueous hydrochloride to pH ˜3. The solution was lyophilized to give crude intermediate L (2 g).

### Preparation of Compound 4

To a solution of intermediate L (0.1 g, 0.26 mmol, purity=58%) in DMF (10 mL) was added intermediate G′ (0.082 g, 0.234 mmol), HATU (0.106 g, 0.28 mmol) and diisopropylethylamine (0.09 g, 0.70 mmol). The mixture was stirred at room temperature overnight. The mixture was diluted with water (20 mL) and extracted with dichloromethane (10 mL×3). The organic layers were dried over Na₂SO₄, filtered and concentrated in vacuum. The residue was purified by high performance liquid chromatography (Waters Xbridge Prep OBD C18 150×30×5μ, 25 ml/min, mobile phase: water (containing 0.05% NH₃.H₂O)/Acetonitrile, gradient from 25/75 to 0/100). The desired fraction was collected and evaporated to remove off acetonitrile in vacuum. The residue was lyophilized to give Compound 4, 0.074 g, 56%.

¹H NMR (400 MHz, CHLOROFORM-d) δ=9.85 (d, J=2.6 Hz, 1H), 8.57 (d, J=2.2 Hz, 1H), 7.29 (s, 2H), 7.08 (d, 0.1=8.4 Hz, 2H), 6.78 (d, J=8.4 Hz, 2H), 6.44 (d, J=8.8 Hz, 2H), 6.11 (br. s., 1H), 4.62 (d, J=5.7 Hz, 2H), 4.52 (quin, J=6.3 Hz, 1H), 4.21 (t, J=7.3 Hz, 2H), 3.87-3.80 (m, 2H), 3.02 (q, J=7.5 Hz, 2H), 2.96 (s, 3H), 1.45 (t, J=7.5 Hz, 3H).

### Synthesis of Compound 5

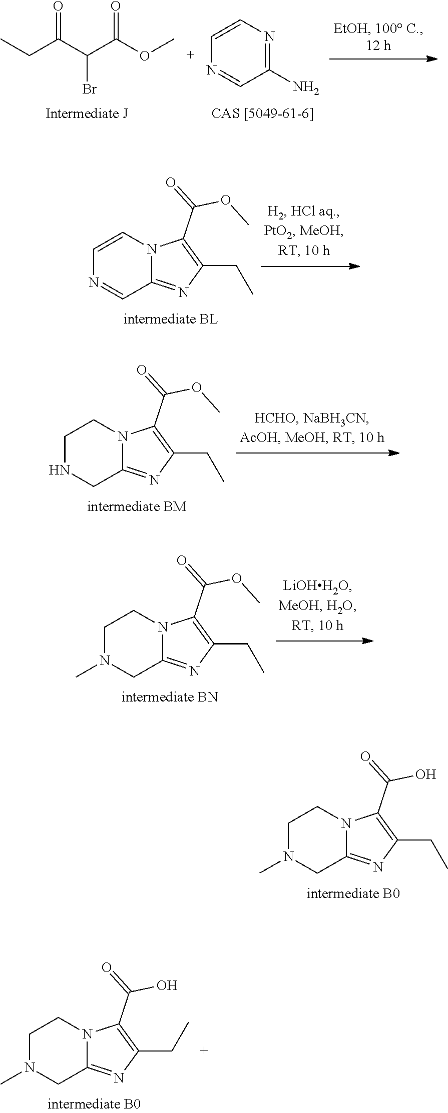

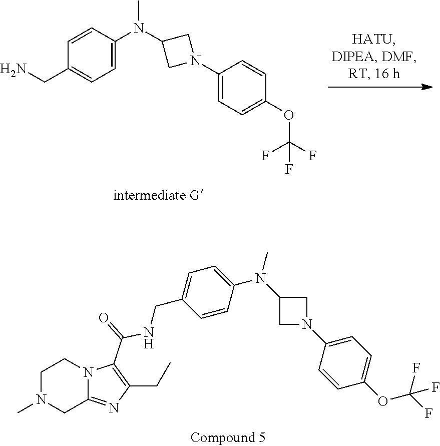 [SMILES: CN(c1ccc(CN)cc1)C1CN(c2ccc(OC(F)(F)F)cc2)C1 ; CCc1nc2n(c1C(=O)NCc1ccc(N(C)C3CN(c4ccc(OC(F)(F)F)cc4)C3)cc1)CCN(C)C2]

### Preparation of intermediate BL

A mixture of 2-aminopyrazine (CAS [5049-61-6], 12 g, 126.18 mmol) and intermediate J (39.6 g, 189.27 mmol) in EtOH (10 mL) was stirred at 100° C. for 12 h. The solvent was removed in vacuum. The crude product was purified by column chromatography (petroleum ether/ethyl acetate=5/1-1/1). The product fractions were collected and the solvent was evaporated to give intermediate BL, 2 g, 8%.

### Preparation of intermediate BM

To a solution of intermediate BL (5 g, 24.36 mmol) in MeOH (20 mL) was added platine dioxide (500 mg) under N₂, followed by addition a drop of con HCl. The suspension was degassed under vacuum and purged with H2 several times. The mixture was stirred under H₂ (15 psi) at 25° C. for 10 hours. The suspension was filtered through a pad of Celite® and the pad was washed with methanol (50 mL). The combined filtrates were concentrated to dryness to give intermediate BM, 5 g, 98%.

### Preparation of intermediate BN

To a solution of intermediate BM (5 g, 23.89 mmol) in MeOH (75 mL) was added formaldehyde aqueous solution (9.7 g, 119.47 mmol, 37%) at 0° C., followed by addition sodium borocyanohydride (7.5 g, 119.47 mmol) and a drop of acetic acid (0.2 mL). Then the mixture was stirred at room temperature for overnight. 10% NH₄Cl solution (25 mL) was added dropwise. The mixture was extracted with ethyl acetate, the combined organic layers were washed with brine, dried over Na₂SO₄, filtered and the solvent was evaporated under vacuum. The residue was purified by column chromatography over silica gel (dichloromethane/methanol=15:1 to 10:1) to give intermediate BN, 1.3 g, 24%.

### Preparation of intermediate BO

To a solution of intermediate BN (0.55 g, 2.46 mmol) in MeOH (25 mL) and water (5 mL) was added lithium hydroxide monohydrate (0.52 g, 12.32 mmol). The mixture was stirred at room temperature for 10 h. The solvent was removed in vacuum to dryness. The residue was purified by high performance liquid chromatography (DuraShell 150×25 mm×5 μm, 25 ml/min, water (containing 0.05% HCl)/Acetonitrile from 100/0 to 70/30). The desired fraction was collected and evaporated to remove off acetonitrile in vacuum. The residue was lyophilized to give intermediate BO, 0.4 g, 78%.

### Preparation of Compound 5

To a solution of intermediate BO (0.045 g, 0.22 mmol) in DMF (20 mL) was added intermediate G′ (0.069 g, 0.196 mmol), HATU (0.097 g, 0.25 mmol) and diisopropylethylamine (0.076 g, 0.58 mmol). The mixture was stirred at room temperature overnight. The mixture was diluted with water (20 mL) and extracted with ethyl acetate (10 mL×3). The organic layers were dried over Na₂SO₄, filtered and concentrated in vacuum. The residue was purified by high performance liquid chromatography (Waters Xbridge Prep OBD C18 150×30×5μ, 25 ml/min, mobile phase: water (containing 0.05% NH₃.H₂O)/acetonitrile, Gradient: from 40/60 to 10/90). The desired fraction was collected and evaporated to remove off acetonitrile in vacuum. The residue was lyophilized to give Compound 5, 0.056 g, 51%.

¹H NMR (400 MHz, CHLOROFORM-d) δ=7.24 (d, J=8.4 Hz, 2H), 7.08 (d, d=8.4 Hz, 2H), 6.76 (d, J=8.8 Hz, 2H), 6.44 (d, J=8.8 Hz, 2H), 5.89 (br. s., 1H), 4.56-4.45 (m, 3H), 4.33 (t, J=5.3 Hz, 2H), 4.20 (t, J=7.3 Hz, 2H), 3.87-3.79 (m, 2H), 3.65 (s, 2H), 2.94 (s, 3H), 2.80 (t, J=5.5 Hz, 2H), 2.72 (q, J=7.5 Hz, 2H), 2.48 (s, 3H), 1.26 (t, J=7.7 Hz, 3H).

### Synthesis of Compound 6

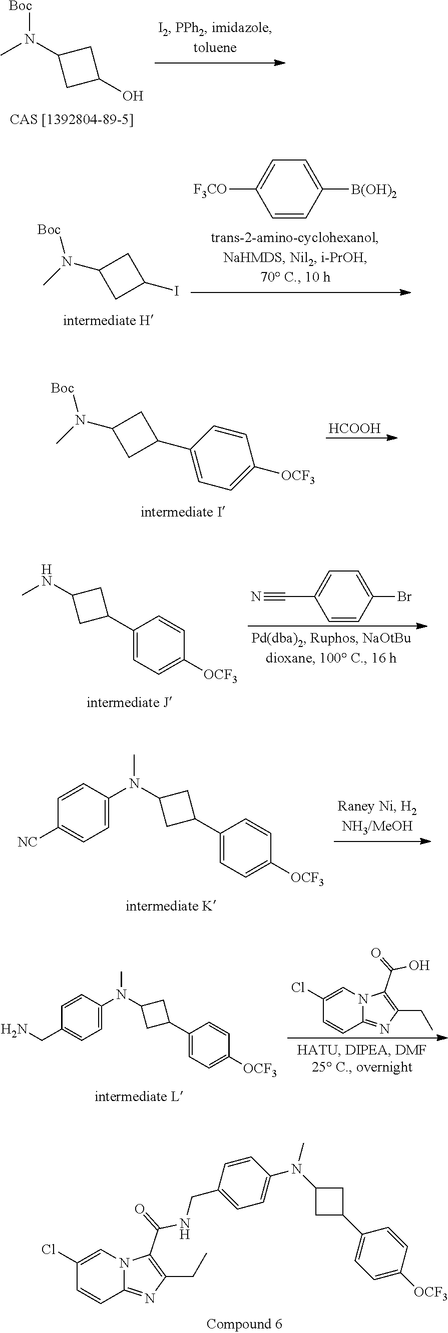

### Preparation of intermediate H′

Triphenylphosphine (18.25 g, 69.56 mmol), imidazole (7.10 g, 104.34 mmol) and iodine (13.24 g, 52.17 mmol) were added to a solution of tert-Butyl N-(3-hydroxycyclobutyl)-N-methylcarbamate (CAS [1392804-89-5], 7 g, 34.78 mmol) in toluene (30 mL). The resulting mixture was refluxed for 1 hour. Ethyl acetate (50 ml) was added and the mixture was washed with water (2×50 mL) and brine (50 mL). The separated organic layer was dried over magnesium sulfate, filtered and the filtrate was concentrated under vacuum. The residue was purified by flash column chromatography over silica gel (eluent: petroleum ether/ethyl acetate 1/0 to 5/1) to give intermediate H′, 8 g, 74%.

### Preparation of intermediate I′

A mixture of (4-(trifluoromethoxy)phenyl)boronic acid (CAS [1399301-27-2], 2.65 g, 12.86 mmol), trans-2-amino-cyclohexanol (0.148 g, 1.28 mmol) and nickel iodine (0.2 g, 0.64 mmol) in isopropanol (30 mL) was stirred at 25° C. for 30 minutes under nitrogen flow. NaHMDS (12.9 mL, 12.86 mmol, 1 M in THF) was added, and the mixture was stirred for 10 minutes under nitrogen flow. Intermediate H′ (2 g, 6.43 mmol) in isopropanol (20 mL) was added and the mixture was stirred at 70° C. for 10 h. The mixture was diluted with dichloromethane (100 mL), washed with water (2×50 mL) and brine (20 mL). The organic layer was dried over sodium sulfate, filtered and concentrated under vacuum. The residue was purified by column chromatography over silica gel (eluent: petroleum ether/ethyl acetate 0 to 10/1) to give intermediate I′, 1.6 g, 72%.

### Intermediate J′

Formic acid (25 mL) was added to intermediate I′ (2 g, 5.79 mmol) at 0° C. under nitrogen atmosphere. The mixture was stirred at 25° C. for 10 hours. The mixture was concentrated under vacuum give intermediate J′, 1.4 g, 98%.

### Intermediate K′

A mixture of intermediate J′ (1.6 g, 6.52 mmol), 4-bromobenzonitrile (CAS [623-00-7], 1.43 g, 7.83 mmol), Pd(dba)₂ (0.37 g, 0.01 mmol), Ruphos (0.609 g, 1.31 mmol) and sodium ter-butoxide (3.14 g, 32.62 mmol) in dioxane (3 mL) was stirred at 100° C. for 16 h under N₂. The mixture was diluted with water (50 mL) and extracted with ethyl acetate (50 mL×3). The combined organic layer was dried over Na₂SO₄, filtered and concentrated in vacuum. The residue was purified by column chromatography (petroleum ether/ethyl acetate=5:1). The product fractions were collected and the solvent was evaporated to give intermediate K′ as pale yellow oil, 1.4 g, 62%.

### Preparation of intermediate L′

To a solution of intermediate K′ (1.54 g, 4.43 mmol) in Ammonia 4M in MeOH (25 mL) was added Raney Nickel (0.01 gg) under N₂. The suspension was degassed under vacuum and purged with H2 several times. The mixture was stirred under H2 (15 psi) at 25° C. for 10 hours. The suspension was filtered through a pad of Celite® and the pad was washed with methanol (80 mL). The combined filtrates were concentrated to give intermediate L′ as yellow oil, 1.4 g, 90%.

### Preparation of Compound 6

To a solution of 6-chloro-2-ethylimidazo[3,2-a]pyridine-3-carboxylic acid (CAS [1216142-18-5], 0.25 g, 1.11 mmol) in DMF (5 mL) was added intermediate L′ (0.3 g, 0.86 mmol), HATU (0.39 g, 1.03 mmol) and diisopropylethylamine (0.332 g, 2.57 mmol). The mixture was stirred at room temperature overnight. The mixture was diluted with water (20 mL) and extracted with dichloromethane (10 mL×3). The organic layers were dried over Na₂SO₄, filtered and concentrated in vacuum. The residue was purified by high performance liquid chromatography (Waters Xbridge Prep OBD C18 150×30×5μ, 25 ml/min, mobile phase: water (containing 0.05% NH₃.H₂O)/Acetonitrile, gradient: 35/65 to 5/95). The desired fraction was collected and evaporated to remove off acetonitrile in vacuum. The residue was lyophilized to give Compound 6, 0.277 g, 58%.

1H NMR (400 MHz, CHLOROFORM-d) δ=9.54 (s, 1H), 7.54 (d, J=9.7 Hz, 1H), 7.37-7.27 (m, 3H), 7.26-7.13 (m, 4H), 6.88-6.76 (m, 2H), 6.02 (br. s., 1H), 4.61 (d, J=5.3 Hz, 2H), 4.17 (quin, J=7.6 Hz, 0.5H), 4.02-3.90 (m, 1H), 3.52 (td. J=4.8, 9.4 Hz, 0.7H), 3.29-3.14 (m, 1H), 2.97 (q, J=7.5 Hz, 2H), 2.93-2.85 (m, 3H), 2.83-2.73 (m, 1.5H), 2.70-2.58 (m, 1H), 2.49 (ddd, J=4.4, 7.7, 12.6 Hz, 1H), 2.24-2.12 (m, 1.5H), 1.40 (t, J=7.5 Hz, 3H)

### Synthesis of Compound 7

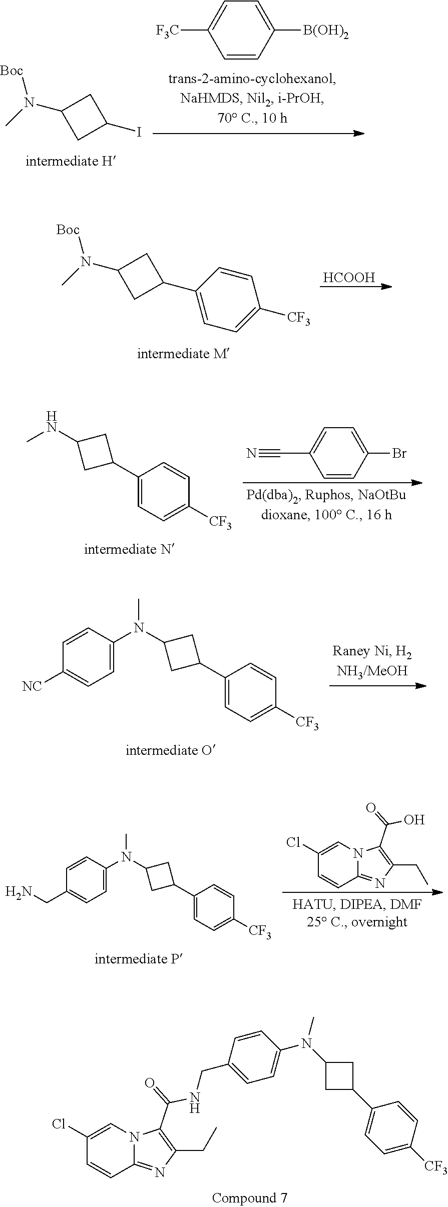

### Preparation of intermediate M′

Accordingly, intermediate M′ was prepared in the same way as intermediate I′ starting from intermediate H′ and 4-(trifluoromethyl)phenyl)boronic acid CAS [128796-39-4] to give 0.22 g, 52%.

### Preparation of Intermediate N′

Accordingly, intermediate N′ was prepared in the same way as intermediate J′ starting from intermediate M′ to give 0.13 g, 82%.

### Preparation of intermediate O′

Accordingly, intermediate O′ was prepared in the same way as intermediate K′ starting from intermediate N′ to give 0.33 g, 26%.

### Preparation of intermediate P′

Accordingly, intermediate P′ was prepared in the same way as intermediate L′ starting from intermediate O′ to give 0.02 g, 100%.

### Preparation of Compound 7

A mixture of 6-chloro-2-ethylimidazo[3,2-a]pyridine-3-carboxylic acid (CAS [1216142-18-5], 0.0135 g, 0.06 mmol), intermediate P′ (0.02 g, 0.06 mmol), HATU (0.03 g, 0.078 mmol) and diisopropylamine (0.023 g, 0.18 mmol) in DMF (4 mL) was stirred at 25° C. for 16 hours. Ethyl acetate (20 mL) was added and the mixture was washed with water (20 mL) and brine (20 mL). The organic layer was dried over magnesium sulfate, filtered and concentrated under vacuum. The residue was purified by column chromatography over silica gel (eluent: petroleum ether/ethyl acetate 1/1 to 0/1) to give F1. F1 was purified by high performance liquid chromatography over Phenomenex Gemini 150×25 mm×10 μm (eluent: 0.5% ammonia water/acetonitrile 26/74 to 0/100). The desired fractions were collected and lyophilized to give F2. F2 was further purified by flash column chromatography over silica gel (eluent: petroleum ether/ethyl acetate 1/1 to 0/1) to give Compound 7, 0.0046 g, 13%.

¹H NMR (400 MHz, CHLOROFORM-d) 6=9.53 (dd, J=0.8, 2.0 Hz, 1H), 7.63-7.58 (m, 0.5H), 7.55 (t, J=8.5 Hz, 2.5H), 7.43 (d, J=8.3 Hz, 0.5H), 7.34 (d, J=8.5 Hz, 1.5H), 7.31-7.27 (m, 2H), 7.26-7.23 (m, 1H), 6.87-6.75 (m, 2H), 6.02 (br. s., 1H), 4.61 (d, J=5.5 Hz, 2H), 4.05-3.94 (m, 1H), 3.35-3.19 (m, 1H), 2.96 (q, J=7.6 Hz, 2H), 2.93-2.86 (m, 3H), 2.84-2.60 (m, 2H), 2.56-2.15 (m, 2H), 1.43-1.37 (m, 3H)

The following compounds were also prepared in accordance with the procedures described herein:

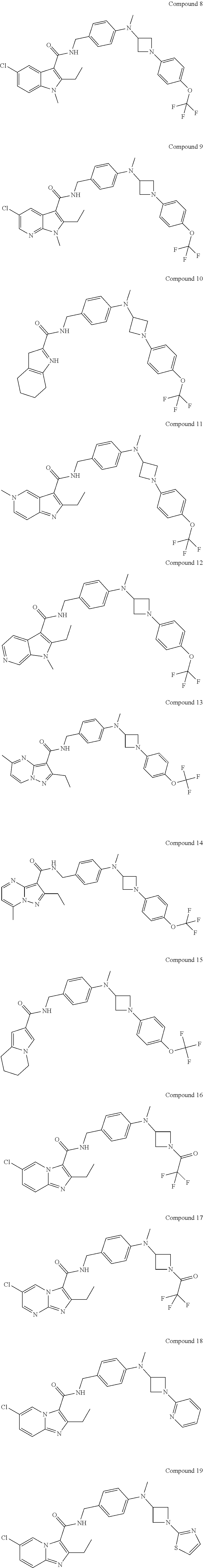

Characterising Data Table

| Com- | Meting Point | LCMS | | | | |
|---|---|---|---|---|---|---|
| pound | (Kofler or | | UV | MW | BPM1/ | LCMS |
| No | DSC) | Rt | Area % | exact | BPM2 | Method |
| Cpd 1 | | 5.25 | 100.0 | 557.2 | 558.2 | Method B |
| Cpd 6 | | 3.22 | 99.8 | 556.2 | 557.2 | Method E |
| Cpd 7 | | 3.27 | 95.2 | 540.2 | 541.2 | Method C |
| Cpd 2 | | 4.07 | 99.6 | 543.2 | 544.1 | Method C |
| Cpd 3 | | 3.51 | 98.8 | 527.3 | 528.2 | Method C |
| Cpd 8 | | 4.8 | 98.9 | 570.2 | 571.1 | Method C |
| Cpd 9 | | 4.73 | 96.8 | 571.2 | 572.1 | Method C |
| Cpd 10 | | 4.42 | 98.2 | 498.2 | 499.2 | Method C |
| Cpd 11 | | 3.64 | 100.0 | 537.2 | 538.2 | Method C |
| Cpd 5 | | 3.52 | 97.2 | 542.3 | 543.2 | Method C |
| Cpd 12 | | 3.59 | 98.7 | 537.2 | 538.2 | Method C |
| Cpd 13 | | 4.4 | 95.1 | 538.2 | 539.1 | Method C |
| Cpd 14 | | 5.48 | 100.0 | 538.2 | 539.3 | Method C |
| Cpd 15 | | 4.15 | 97.4 | 498.2 | 499.1 | Method C |
| Cpd 4 | | 4.25 | 99.9 | 558.2 | 559.1 | Method C |
| Cpd 16 | | 4.92 | 95.7 | 493.1 | 494.1 | Method D |
| Cpd 17 | | 3.59 | 89.0 | 494.1 | 495.1 | Method E |
| Cpd 18 | 158.86° C./ | 2.96 | 100.0 | 474.2 | 475.1/ | Method A |
| | −98.88 Jg-1 | | | | 473.3 | |
| | 25° C. to | | | | | |
| | 350° C./10° C. | | | | | |
| | min/40 μl Al | | | | | |
| Cpd 19 | 152.57° C./ | 2.93 | 96.4 | 480.2 | 481.1/ | Method A |
| | −97.89 Jg-1 | | | | 479.3 | |
| | 25° C. to | | | | | |
| | 350° C./10° C. | | | | | |
| | min/40 μl Al | | | | | |

Analytical Methods

LCMS

The mass of some compounds was recorded with LCMS (liquid chromatography mass spectrometry). The methods used are described below.

General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl . . . ), the reported value is the one obtained for the lowest isotope mass. All results were obtained with experimental uncertainties that are commonly associated with the method used.

Hereinafter, “SQD” means Single Quadrupole Detector, “RT” room temperature, “BEH” bridged ethylsiloxane/silica hybrid, “HSS” High Strength Silica, “DAD” Diode Array Detector.

TABLE
LCMS Method codes (Flow expressed in mL/min; column
temperature (T) in ° C.; Run time in minutes).

| Method | | | Mobile | | Flow | Run |
|---|---|---|---|---|---|---|
| code | Instrument | Column | phase | gradient | Column T | time |
| Method | Waters: | Waters: BEH | A: 95% | 84.2% A for 0.49 min, | 0.343 | 6.2 |
| A | Acquity | C18 (1.7 μm, | CH₃COONH₄ | to 10.5% A in | 40 | |
| | UPLC ®- | 2.1 × 100 mm) | 7 mM/5% | 2.18 min, held for | 40 | |
| | DAD and | | CH₃CN, B: | 1.94 min, back to | | |
| | Quattro | | CH₃CN | 84.2% A in 0.73 min, | | |
| | Micro ™ | | | held for 0.73 min. | | |

Hereinafter, “MSD” Mass Selective Detector, “DAD” Diode Array Detector.

TABLE
LCMS Method codes (Flow expressed in mL/min;
column temperature (T) in ° C.; Run time in minutes).

| Method | | | | | Flow | Run |
|---|---|---|---|---|---|---|
| Code | Instrument | Column | Mobile phase | gradient | Column T | time |
| Method B | Agilent: | Agilent TC- | A: CF₃COOH | 100% A for 1 min, | 0.8 | 10.5 |
| | 1100/1200- | C18 (5 μm, | 0.1% in water, | to 40% A in | 50 | |
| | DAD and | 2.1 × 50 mm) | B: CF₃COOH | 4 min, to 15% A in | | |
| | MSD | | 0.05% in | 2.5 min, back to | | |
| | | | CH₃CN | 100% A in 2 min. | | |
| Method C | Agilent: | Agilent TC- | A: CF₃COOH | 90% A for | 0.8 | 10.5 |
| | 1100/1200- | C18 (5 μm, | 0.1% in water, | 0.8 min, to 20% A | 50 | |
| | DAD and | 2.1 × 50 mm) | B: CF₃COOH | in 3.7 min, held | | |
| | MSD | | 0.05% in | for 3 min, back to | | |
| | | | CH₃CN | 90% A in 2 min. | | |
| Method D | Agilent: | Waters: | A: NH₄OH | 100% A for 1 min, | 0.8 | 10.5 |
| | 1100/1200- | XBridge ™ | 0.05% in water, | to 40% A in | 40 | |
| | DAD and | Shield RP18 | B: CH₃CN | 4 min, held for | | |
| | MSD | (5 μm, | | 2.5 min, back to | | |
| | | 2.1 × 50 mm) | | 100% A in 2 min. | | |
| Method E | Agilent: | Phenomenex: | A: CF₃COOH | 90% A for | 0.8 | 10 |
| | 1200-DAD | Luna-C18 | 0.1% in water, | 0.8 min, to 20% A | 50 | |
| | and | (5 μm, | B: CF₃COOH | in 3.7 min, held | 50 | |
| | MSD6110 | 2 × 50 mm) | 0.05% in | for 3 min, back to | | |
| | | | CH₃CN | 90% A in 2 min. | | |

When a compound is a mixture of isomers which give different peaks in the LCMS method, only the retention time of the main component is given in the LCMS table.

Pharmacological Examples

MIC Determination for Testing Compounds Against M. tuberculosis.

Test 1

Appropriate solutions of experimental and reference compounds were made in 96 well plates with 7H9 medium. Samples of Mycobacterium tuberculosis strain H37Rv were taken from cultures in logarithmic growth phase. These were first diluted to obtain an optical density of 0.3 at 600 nm wavelength and then diluted 1/100, resulting in an inoculum of approximately 5×10 exp5 colony forming units per well. Plates were incubated at 37° C. in plastic bags to prevent evaporation. After 7 days, resazurin was added to all wells. Two days later, fluorescence was measured on a Gemini EM Microplate Reader with 543 excitation and 590 nm emission wavelengths and MIC₅₀ and/or pIC₅₀ values (or the like, e.g. IC₅₀, IC₉₀, pIC₉₀, etc) were (or may be) calculated.

Test 2

Round-bottom, sterile 96-well plastic microtiter plates are filled with 100 μl of Middlebrook (1×) 7H9 broth medium. Subsequently, an extra 100 μl medium is added to column 2. Stock solutions (200× final test concentration) of compounds are added in 2 μl volumes to a series of duplicate wells in column 2 so as to allow evaluation of their effects on bacterial growth. Serial 2-fold dilutions are made directly in the microtiter plates from column 2 to 11 using a multipipette. Pipette tips are changed after every 3 dilutions to minimize pipetting errors with high hydrophobic compounds. Untreated control samples with (column 1) and without (column 12) inoculum are included in each microtiter plate. Approximately 10000 CFU per well of Mycobacterium tuberculosis (strain H37RV), in a volume of 100 μl in Middlebrook (1×) 7H9 broth medium, is added to the rows A to H, except column 12. The same volume of broth medium without inoculum is added to column 12 in row A to H. The cultures are incubated at 37° C. for 7 days in a humidified atmosphere (incubator with open air valve and continuous ventilation). On day 7 the bacterial growth is checked visually.

The 90% minimal inhibitory concentration (MIC₉₀) is determined as the concentration with no visual bacterial growth.

TEST 3: Time Kill Assays

Bactericidal or bacteriostatic activity of the compounds can be determined in a time kill assay using the broth dilution method. In a time kill assay on Mycobacterium tuberculosis (strain H37RV), the starting inoculum of M. tuberculosis is 10⁶ CFU/ml in Middlebrook (lx) 7H9 broth. The antibacterial compounds are used at the concentration of 0.1 to 10 times the MIC₉₀. Tubes receiving no antibacterial agent constitute the culture growth control. The tubes containing the microorganism and the test compounds are incubated at 37° C. After 0, 1, 4, 7, 14 and 21 days of incubation samples are removed for determination of viable counts by serial dilution (10⁻¹ to 10⁻⁶) in Middlebrook 7H9 medium and plating (100 μl) on Middlebrook 7H11 agar. The plates are incubated at 37° C. for 21 days and the number of colonies are determined. Killing curves can be constructed by plotting the log₁₀ CFU per ml versus time. A bactericidal effect is commonly defined as 3-log₁₀ decrease in number of CFU per ml as compared to untreated inoculum. The potential carryover effect of the drugs is removed by serial dilutions and counting the colonies at highest dilution used for plating.

TEST 4 (see also test 1 above; in this test a different strain of Mycobacterium tuberculosis strain is employed)

Appropriate solutions of experimental and reference compounds were made in 96 well plates with 7H9 medium. Samples of Mycobacterium tuberculosis strain EH 4.0 (361.269) were taken from cultures in stationary growth phase. These were first diluted to obtain an optical density of 0.3 at 600 nm wavelength and then diluted 1/100, resulting in an inoculum of approximately 5×10 exp5 colony forming units per well. Plates were incubated at 37° C. in plastic bags to prevent evaporation. After 7 days, resazurin was added to all wells. Two days later, fluorescence was measured on a Gemini EM Microplate Reader with 543 nm excitation and 590 nm emission wavelengths and MIC50 and/or pIC50 values (or the like, e.g. IC50, IC90, pIC90, etc) were (or may be) calculated. pIC50 values may be recorded below in μg/mL.

Results

Compounds of the invention/examples, for example when tested in Test 1 or Test 2 described above, may typically have an IC₉₀ value from 0.01 to 10 μg/ml. Compounds of the invention/examples, for example when tested in Test 1 or Test 2 described above, may typically have a pIC₅₀ from 3 to 10 (e.g. from 4.0 to 9.0, such as from 5.0 to 8.0)

Compounds of the examples were tested in Test 1 described above (in section “Pharmacological Examples”) and the following results were obtained:

Biological Data Table
Compounds of the examples were tested in Test 4 described
above (in section “Pharmacological Examples”) and
the following results were obtained:

| Compound No | pIC₅₀ |
|---|---|
| Cpd 1 | 8 |
| Cpd 6 | 7.7 |
| Cpd 7 | 7.4 |
| Cpd 2 | 8 |
| Cpd 3 | 7.65 |
| Cpd 8 | <4.8 |
| Cpd 9 | <4.8 |
| Cpd 10 | <4.9 |
| Cpd 11 | 5.45 |
| Cpd 5 | 6.9 |
| Cpd 12 | <4.9 |
| Cpd 13 | 5.1 |
| Cpd 14 | <4.8 |
| Cpd 15 | <4.8 |
| Cpd 4 | 7.9 |
| Cpd 16 | |
| Cpd 17 | |
| Cpd 18 | |
| Cpd 19 | |

1. A compound of formula (I) 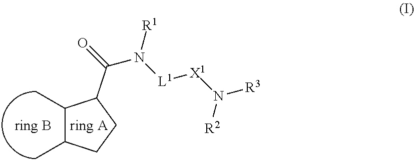
wherein
R¹ represents C₁₋₆ alkyl or hydrogen;
L¹ represents a linker group —C(R^{a})(R^{b})—;
X¹ represents an optional carbocyclic aromatic linker group (which linker group may itself be optionally substituted by one or more substituents selected from fluoro, —OH, —OC₁₋₆ alkyl and C₁₋₆ alkyl, wherein the latter two alkyl moieties are themselves optionally substituted by one or more fluoro atoms);
R^{a} and R^{b} independently represent hydrogen or C₁₋₆ alkyl (optionally substituted by one or more fluoro atoms);
R² and R³ independently represent:
(i) C₁₋₃ alkyl optionally substituted by one or more substituents selected from Q¹ and ═O; or
(ii) cycloalkyl or heterocycloalkyl (e.g. a 4-6-membered ring containing a nitrogen atom, so forming e.g. an azetidinyl group), each of which is optionally substituted by one or more substituents selected from Q³ and ═O;
Q¹ and Q³ each independently represent one or more substituents selected from:
aryl (e.g. phenyl) optionally substituted by one or more substituents selected from halo, C₁₋₆ alkyl and —OC₁₋₆ alkyl (which latter two alkyl moieties may themselves be substituted with one or more fluoro atoms)
heteroaryl (e.g. a 5- or 6-membered heteroaryl group containing one or two heteroatoms, so forming e.g. a pyridinyl or thiazolyl group) optionally substituted as defined herein (but in an aspect, such heteroaryl groups are unsubstituted)
C₁₋₆ alkyl (e.g. C₁₋₃ alkyl) optionally substituted by one or more substituents selected from ═O and fluoro (e.g. so forming a —C(O)—CF₃ group)
ring A is a 5-membered aromatic ring containing at least one heteroatom (preferably containing at least one nitrogen atom);
ring B is a 5- or 6-membered ring, which may be aromatic or non-aromatic, optionally containing one to four heteroatoms (preferably selected from nitrogen, oxygen and sulfur);
either ring A and/or ring B may be optionally substituted by one or more substituents selected from: halo, C₁₋₆ alkyl (optionally substituted by one or more halo, e.g. fluoro atoms) and/or —OC₁₋₆alkyl (itself optionally substituted by one or more fluoro atoms),
or a pharmaceutically-acceptable salt thereof.

2. A compound of formula (I) 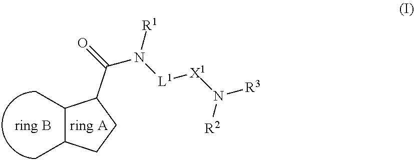
wherein
R¹ represents C₁₋₆ alkyl or hydrogen;
L¹ represents a linker group —C(R^{a})(R^{b})—;
X¹ represents an optional carbocyclic aromatic linker group (which linker group may itself be optionally substituted by one or more substituents selected from fluoro, —OH, —OC₁₋₆ alkyl and C₁₋₆ alkyl, wherein the latter two alkyl moieties are themselves optionally substituted by one or more fluoro atoms);
R^{a} and R^{b} independently represent hydrogen or C₁₋₆ alkyl (optionally substituted by one or more fluoro atoms);
R² and R³:
(i) independently represent C₁₋₆ alkyl optionally substituted by one or more substituents selected from Q¹ and ═O;
(ii) independently represent aryl or heteroaryl, each of which is optionally substituted by one or more substituents selected from Q²; or
(iii) independently represent cycloalkyl or heterocycloalkyl, each of which is optionally substituted by one or more substituents selected from Q³ and ═O;
Q¹, Q² and Q³ each independently represent one or more substituents selected from halo, C₁₋₆ alkyl, —OC₁₋₆ alkyl (which latter two alkyl moieties may themselves be optionally substituted by one or more substituents selected from ═O and halo, e.g. fluoro, atoms), aryl and heteroaryl (which latter two aromatic groups may themselves be optionally substituted by one or more substituents selected from halo, C₁₋₆ alkyl and —OC₁₋₆ alkyl, which latter two alkyl moieties may themselves be substituted with one or more fluoro atoms);
ring A is a 5-membered aromatic ring containing at least one heteroatom (preferably containing at least one nitrogen atom);
ring B is a 5- or 6-membered ring, which may be aromatic or non-aromatic, optionally containing one to four heteroatoms (preferably selected from nitrogen, oxygen and sulfur);
either ring A and/or ring B may be optionally substituted by one or more substituents selected from: halo, C₁₋₆ alkyl (optionally substituted by one or more halo, e.g. fluoro atoms) and/or —OC₁₋₆alkyl (itself optionally substituted by one or more fluoro atoms),
or a pharmaceutically-acceptable salt thereof.

3. A compound as claimed in claim 1 or claim 2, wherein:
R¹ represents hydrogen;
R^{a} and R^{b} independently represent hydrogen; and/or
L¹ represents —CH₂—.

4. A compound as claimed in claim 1 or claim 2, wherein when X¹ represents a carbocyclic aromatic linker group that is:
-phenylene- (especially a 1,4-phenylene), e.g.: 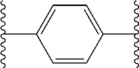 [SMILES: *c1ccc(*)cc1]
-naphthylene, e.g.:

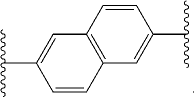 [SMILES: *c1ccc2cc(*)ccc2c1]

5. A compound as claimed in claim 1, wherein:
R² and R³:
(i) independently represent C₁₋₃ alkyl optionally substituted by one or more substituents selected from Q¹ and ═O;
(ii) independently represent cycloalkyl or heterocycloalkyl, each of which is optionally substituted by one or more substituents selected from Q³ and ═O; and/or
Q¹, Q² and Q³ each independently represent one or more substituents selected from aryl (e.g. phenyl) optionally substituted by one or more substituents selected from halo, C₁₋₆ alkyl and —OC₁₋₆ alkyl (which latter two alkyl moieties may themselves be substituted with one or more fluoro atoms).

6. A compound as claimed in claim 1 wherein:
ring A is represented as follows: 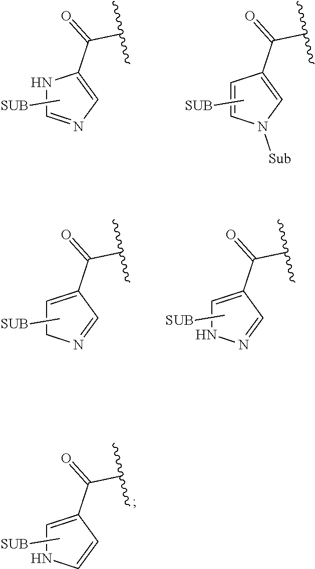
and/or
ring B is represented as follows: 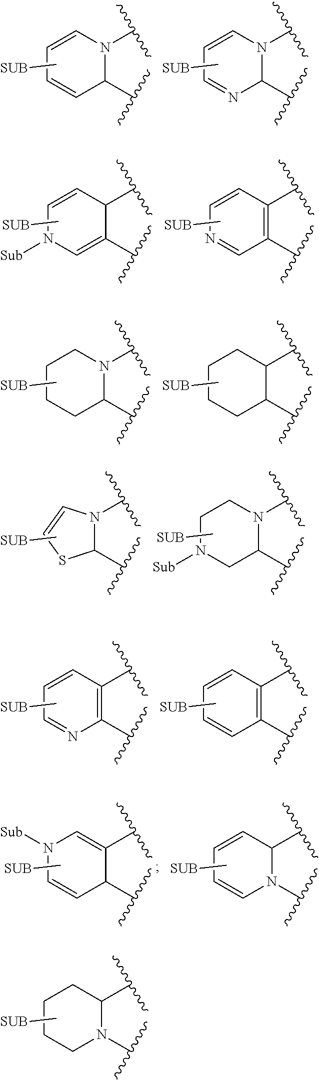
wherein “SUB” and “Sub” represent one or more possible substituents on the relevant atom (e.g. carbon or nitrogen atom).

7. A compound as claimed in claim 1, wherein the combined ring systems, i.e. Ring A and Ring B may be represented as follows: 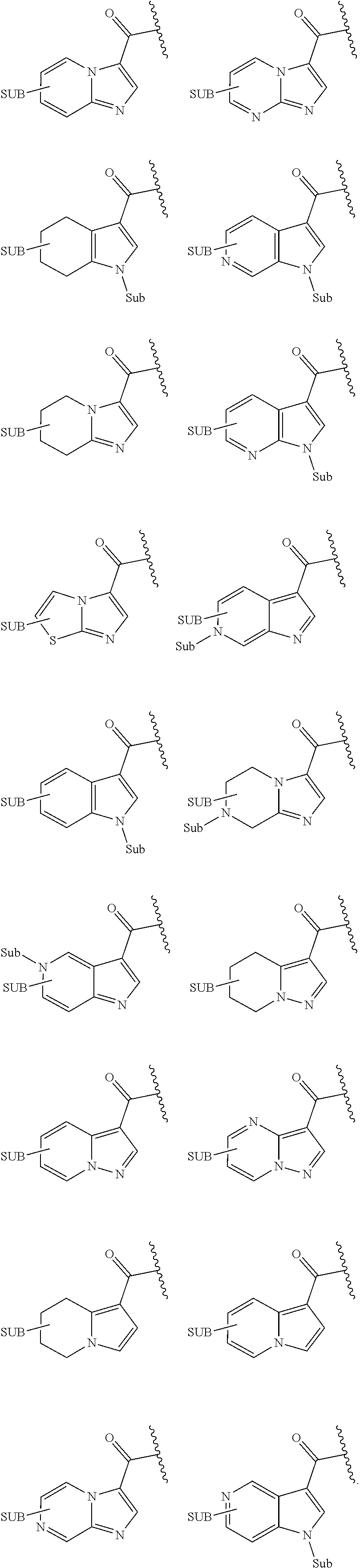
where “SUB” represents one or more possible substituents on the bicycle (i.e. on ring A and/or on ring B) and “Sub” represents a possible optional substituent on the N atom of the bicycle (unsubstituted in this context would mean “NH”).

8. A compound of claim 1 wherein:
L¹ represents —CH₂—;
one of R² and R³ represents:
cycloalkyl or heterocycloalkyl (e.g. a 4-6-membered ring containing a nitrogen atom, so forming e.g. an azetidinyl group), each of which is optionally substituted by one or more substituents selected from Q³ and ═O; and
the other (one of R² or R³) represents C₁₋₆ (e.g. C₁₋₃ alkyl) optionally substituted by one or more substituents selected from Q¹ and ═O.

9. A compound as claimed in claim 8, wherein:
when R² or R³ represents cycloalkyl or heterocycloalkyl, then such cyclic groups are substituted by at least one substituent selected from Q³;
Q³ represents aryl or heteroaryl, both of which are optionally substituted as defined in claim 1.

10. A compound as claimed in claim 8 or claim 9, wherein:
ring A and ring B together represent a 8 or 9-membered bicyclic ring (ring A is a 5-membered ring and ring B may be a 5 or 6-membered ring, in which both rings are preferably aromatic) containing at least one nitrogen atom (and in a major embodiment, at least one nitrogen atom that is common to both rings);
optional substituents on ring A and ring B are halo, C₁₋₃ alkyl and —OC₁₋₃ alkyl; and
other integers are as defined herein.

11. (canceled)

12. A pharmaceutical composition comprising a pharmaceutically acceptable carrier and, as active ingredient, a therapeutically effective amount of a compound as defined in claim 1.

13. (canceled)

14. A method for treating a patient with tuberculosis, said method comprising administering to said patient a therapeutically effective amount of a compound according to claim 1.

15. A method of treatment of a bacterial infection, which method comprises administration of a therapeutically effective amount of a compound according to claim 1.

16. A combination of (a) a compound according to claim 1, and (b) one or more other anti-tuberculosis agent.

17. A product containing (a) a compound according to claim 1, and (b) one or more other anti-tuberculosis agent, as a combined preparation for simultaneous, separate or sequential use in the treatment of a bacterial infection.

18. A process for the preparation of a compound of formula (I) as claimed in claim 1, which process comprises:
(i) reaction of a compound of formula (II), 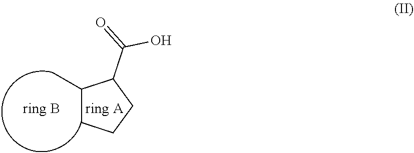
wherein the integers are as defined in claim 1, or a suitable derivative thereof, with a compound of formula (III), 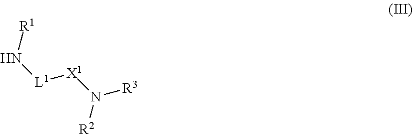
wherein the integers are as defined in claim 1;
(ii) coupling of a compound of formula (IV), 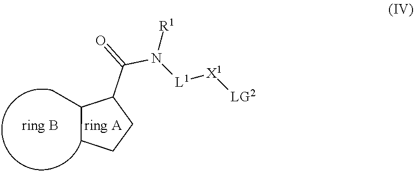
wherein the integers are as defined in claim 1, and LG² represents a suitable leaving group, with a compound of formula (V),
HN(R²)(R³) (V)
wherein the integers are as defined in claim 1.
